(19) United States
(12) Patent Application Publication
(10) Pub. No.: US 2015/0025386 A1
(43) Pub. Date: Jan. 22, 2015

(54) PORTABLE DIAGNOSTIC ULTRASOUND APPARATUS

(71) Applicant: HITACHI ALOKA MEDICAL, LTD., Mitaka-shi, Tokyo (JP)

(72) Inventors: Keigo Ninomiya, Mitaka-shi (JP); Masa Yamamoto, Mitaka-shi (JP); Miyuki Kaga, Mitaka-shi (JP)

(73) Assignee: HITACHI ALOKA MEDICAL, LTD., Mitaka-shi, Tokyo (JP)

(21) Appl. No.: 14/380,885

(22) PCT Filed: Feb. 4, 2013
(86) PCT No.: PCT/JP2013/052493

§ 371 Date: Aug. 25, 2014

(30) Foreign Application Priority Data
Feb. 27, 2012 (JP) 2012-040517

(51) Int. Cl. A61B 8/00

(52) U.S. Cl. CPC A61B 8/462; A61B 8/54; A61B 8/4427
USPC 600/443

(57) ABSTRACT

In order to provide technology for limiting the deterioration of hardware in a portable diagnostic ultrasound apparatus without increasing user workload, internal processing of the portable diagnostic ultrasound apparatus is controlled according to the opening or closing of the cover case in which the monitor is disposed. In particular, when the cover case is closed, not only is output of ultrasonic waves stopped but processing that is being executed inside the apparatus is appropriately terminated and the power is turned off. When so doing, processing that is being executed is interrupted and cancelled and processing that is waiting to be executed is cancelled. The cancelled information is stored in a storage device.

### TECHNICAL FIELD

The present invention relates to a portable diagnostic ultrasound apparatus, and more particularly to a control technology dealing with opening/closing of a monitor.

### BACKGROUND ART

A diagnostic ultrasound apparatus can display a tomographic image of a soft tissue of a living body and an image of a blood flow flowing through the living body and the like on a monitor for observation on a substantially real time basis and is widely used in the medical field. For a visiting examination on a visit to a patient, a diagnosis at the bedside of an inpatient and the like, portable models with batteries mounted, including a hand-carried type, a hand-held type and the like, have spread. The portable model diagnostic ultrasound apparatus generally has a configuration of a so-called laptop type computer including a main body housing, and a lid housing provided with a monitor (see Patent Document 1, for example). With such laptop computer type diagnostic ultrasound apparatus (hereinafter referred to as a “portable diagnostic ultrasound apparatus”), the lid housing is opened when in use, and treatment is conducted while the monitor is observed. Moreover, while not in use, the lid housing is closed, and the monitor is accommodated in the body housing.

In general, the diagnostic ultrasound apparatus is provided with a function called automatic freeze for automatically stopping emission of ultrasonic waves when an operation has not been performed for a predetermined period of time (see Patent Document 2, for example). This is to prevent deterioration of an ultrasonic probe that would otherwise be caused by continuous ultrasonic wave outputs.

### CITATION LIST

### Patent Literature

Patent Document 1: Japanese Patent Laid-Open Publication No. 2011-72584
Patent Document 2: Japanese Patent Laid-Open Publication No. Hei 1-68239

### SUMMARY OF INVENTION

### Technical Problem

In the diagnostic ultrasound apparatus, various types of processing are executed other than output of ultrasonic waves from the ultrasonic probes. Thus, even if the output of the ultrasonic waves from the ultrasonic probe is stopped by the automatic freezing function, many units of hardware other than the ultrasonic probes continue operating. In the case of the portable diagnostic ultrasound apparatus described in Patent Document 1, since the monitor is accommodated in the main body, a state of a power supply of the portable diagnostic ultrasound apparatus and whether or not all the ongoing processing has been ended cannot be checked easily from a video image outputted to the monitor. As a result, the apparatus is left in a powered state, and deterioration of the hardware can progress faster.

The present invention was conceived in view of the above described circumstances, and an advantage of the present invention is to provide a technology of suppressing deterioration of hardware without increasing user workload in a portable diagnostic ultrasound apparatus.

### Solution to Problem

The present invention controls processing inside a portable diagnostic ultrasound apparatus in accordance with opening/closing of a lid housing in which a monitor is arranged in the portable diagnostic ultrasound apparatus. Particularly, when the lid housing is closed, not only is output of ultrasonic waves stopped but also the ongoing processing in the apparatus is ended appropriately, and the apparatus is turned off.

At this time, the ongoing processing is interrupted and cancelled, and the processing waiting to be executed is cancelled. The cancelled information is stored in a storage device.

More specifically, there is provided a portable diagnostic ultrasound apparatus provided with a main body housing including an ultrasonic transmission/receipt unit for generating ultrasonic waves and transmitting them to an ultrasonic probe and for receiving the ultrasonic waves from the ultrasonic probe, an ultrasonic image forming unit for forming an ultrasonic image on the basis of the ultrasonic waves received in the ultrasonic transmission/receipt unit, and a control unit; and a lid housing including a display device for displaying the ultrasonic image and attached to the main body housing capable of being opened/closed, in which the control unit is provided with an automatic end portion for ending the portable diagnostic ultrasound apparatus when the lid housing is closed and a safe end portion for executing safe end processing for safely ending ongoing processing and processing scheduled to be executed in the portable diagnostic ultrasound apparatus.

### Advantageous Effects of Invention

According to the present invention, in the portable diagnostic ultrasound apparatus, deterioration of hardware can be suppressed without increasing user workload.

### BRIEF DESCRIPTION OF DRAWINGS

FIG. 1(a) is an appearance view of a portable diagnostic ultrasound apparatus of a first embodiment, and FIG. 1(b) is a block diagram of the portable diagnostic ultrasound apparatus of the first embodiment.

FIG. 2 is a functional block diagram of a control unit of the first embodiment.

FIG. 3 is a flowchart of automatic end processing of the first embodiment.

FIG. 4 is a flowchart of safe end processing of the first embodiment.

FIG. 5 is a flowchart of start processing after automatic end of the first embodiment.

FIG. 6 is a flowchart of automatic end processing of a second embodiment.

FIG. 7 is a flowchart of examination information storing processing of the second embodiment.

FIG. 8 is a flowchart of start processing after automatic end of the second embodiment.

FIG. 9 is a block diagram of a portable diagnostic ultrasound apparatus of a third embodiment.

FIG. 10 is a functional block diagram of a control unit of the third embodiment.

FIG. 11 is a flowchart of automatic end processing of the third embodiment.

FIG. 12 is a functional block diagram of a control unit of a fourth embodiment.

FIG. 13 is a flowchart of automatic end processing of the fourth embodiment.

FIG. 14 is a flowchart of automatic end processing of a fifth embodiment.

FIG. 15 is a functional block diagram of a control unit of a sixth embodiment.

FIG. 16 is a flowchart of automatic end processing of the sixth embodiment.

FIG. 17 is a flowchart of transfer preparation processing of the sixth embodiment.

FIGS. 18(a) and 18(b) are explanatory diagrams for explaining example warning messages of the sixth embodiment.

FIG. 19 is a flowchart of a reset processing related portion of a variation of the automatic end processing of the embodiment of the present invention.

FIG. 20 is a flowchart of a variation of the automatic end processing of the embodiment of the present invention.

### DESCRIPTION OF EMBODIMENTS

### First Embodiment

A first embodiment to which the present invention is applied will be described. Throughout the specification, the same reference numerals are assigned to those elements having the same functions, and repetitious explanation will be omitted. In this embodiment, in a portable diagnostic ultrasound apparatus to which a monitor which is a display device is attached capable of being opened/closed, electric conduction to the apparatus is automatically stopped safely after a predetermined period of time elapses since the monitor is closed. At this time, ongoing processing is stored so that it can be resumed at restart.

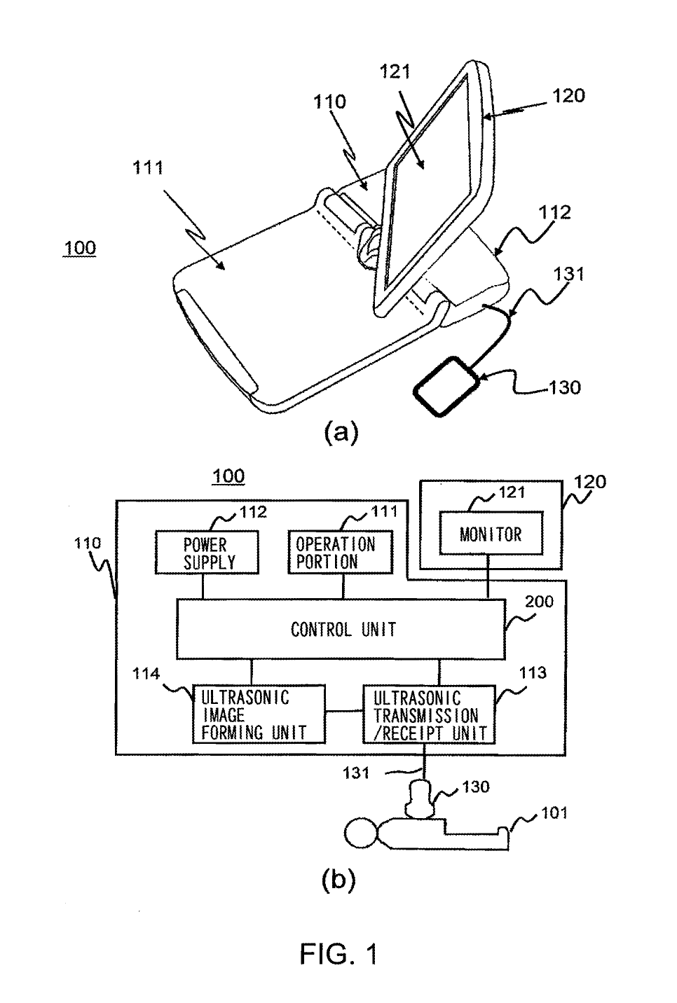

First, a portable diagnostic ultrasound apparatus 100 of this embodiment will be explained by reference to FIGS. 1(a) and 1(b). FIG. 1(a) is an appearance view of the portable diagnostic ultrasound apparatus 100, and FIG. 1(b) is a block diagram of the portable diagnostic ultrasound apparatus 100. The portable diagnostic ultrasound apparatus 100 constitutes a two-dimensional ultrasonic image, a three-dimensional ultrasonic image, or various Doppler images of a portion to be diagnosed by using an echo signal obtained by transmitting and receiving ultrasonic waves into a subject 101, and displays the resultant signals. As illustrated in FIG. 1(a), the portable diagnostic ultrasound apparatus 100 as a structural body is provided with a processing device having major processing functions, an operation portion (control panel) 111 which is an interface for receiving an instruction from an operator, a main body housing 110 accommodating a power supply 112 for supplying electricity to each portion, a lid housing 120 including a monitor 121 for displaying an image, information, messages and the like, and an ultrasonic probe 130 for transmitting ultrasonic waves to the subject 101 and receiving a reflected echo. The ultrasonic probe 130 is connected to the main body housing 110 through a cable 131.

Moreover, the lid housing 120 provided with the monitor 121 is attached to the main body housing 110, in such a way as to be capable of being opened/closed. The monitor 121 is arranged so as to be located on the main body housing 110 side when the lid housing 120 is closed. Therefore, in this embodiment, when the lid housing 120 is closed, the monitor 121 for checking a video output is placed down so as to face the operation portion 111, and it becomes difficult to visually check instantaneously whether or not the portable diagnostic ultrasound apparatus 100 is in a conduction state.

The processing device generates a pulse-state electric signal for generating an ultrasonic signal to be transmitted to the subject 101 and is provided with an ultrasonic transmission/receipt unit 113 for transmitting it to the ultrasonic probe 130 and for converting the echo signal received by the ultrasonic probe 130 to an electric signal, an ultrasonic image forming unit 114 for forming a two-dimensional ultrasonic image, a three-dimensional ultrasonic image, or various Doppler images from the electric signal received by the ultrasonic transmission/receipt unit 113, and a control unit 200 for controlling each portion. The image formed in the ultrasonic image forming unit 114 is displayed on the monitor 121.

The processing device is provided with a CPU, a memory, and a storage device and realizes processing of each of the ultrasonic transmission/receipt unit 113, the ultrasonic image forming unit 114, and the control unit 200 by loading a program stored in the storage device and executing it by the CPU.

The ultrasonic probe 130 is formed by aligning an array of a plurality of oscillator elements for 1 to m channels in a long axis direction of the ultrasonic probe. The ultrasonic transmission/receipt unit 113 is to supply a wave transmission pulse to the ultrasonic probe 130 and to process the received echo signal as described above and is provided therein with a transmission portion for transmitting an ultrasonic beam by controlling the ultrasonic probe 130, a receipt portion for receiving the echo signal from the inside of the subject 101 of the transmitted ultrasonic beam, a complex signal conversion portion for applying quadrature detection to the echo signal received by the reception portion and converting it to a complex signal, and an ultrasonic transmission/receipt control unit for controlling each of these portions.

The ultrasonic image forming unit 114 is to form an ultrasonic tomographic image by using the complex signal converted in the complex signal conversion portion of the ultrasonic transmission/receipt unit 113 and is provided with an ultrasonic image information generating portion for generating ultrasonic image information of an examination target by using the complex signal, a digital scan converter portion (hereinafter referred to as a DSC portion) for scanning/converting the generated ultrasonic image information to a TV display image pattern and generating ultrasonic image data, a graphic data generating portion for generating graphic data such as a scale, marks, characters, and the like to be added to the image on the basis of the image data obtained by scanning/conversion in the DSC portion, a synthesis storage portion for synthesizing and storing the ultrasonic image data generated in the DSC portion and the graphic data generated in the graphic data generating portion, and an interface of the ultrasonic image forming unit which is an interface for reading out initial values, control parameters, and the like required for various types of processing of the ultrasonic image information generating portion for generating the ultrasonic image information, the DSC portion, the graphic data generating portion, and the synthesis storage portion in accordance with an instruction from the control unit 200 and setting them for the ultrasonic image information generating portion, the DSC portion, the graphic data generating portion, and the synthesis storage portion.

The monitor 121 is constituted by a CRT monitor, a liquid crystal monitor or the like, for example. The operation portion 111 is provided with a keyboard having various keys, a trackball, and the like.

The power supply 112 supplies AC power, when an AC cable is connected, and supplies power from a battery to the apparatus when the AC cable is not connected. The battery not only supplies power but also notifies the control unit 200 of information relating to the battery such as a remaining battery capacity, an estimated usable time, and the like.

Any one of the lid housing 120, the main body housing 110, the monitor 121, and the operation portion 111 is provided with a sensor (not shown) for detecting that the lid housing 120 is closed, and the monitor 121 is accommodated in the main body 110. When the sensor detects that the lid body is closed, the sensor outputs a closed signal to the control unit 200, whereas when the sensor detects that the lid body is opened, it outputs an open signal to the control unit 200. The sensor may be constituted so as to output a signal indicating whether the lid housing 120 is closed or opened at present to the control unit 200 every predetermined period of time.

A storage portion 230 is composed of a storage device such as a hard disk and the like, a temporary storage memory RAM, and the like.

Subsequently, the control unit 200 of this embodiment will be described.

The control unit 200 of this embodiment controls an operation of each portion of the portable diagnostic ultrasound apparatus 100 in accordance with an instruction from the operation portion 111 or settings stored in the storage device in advance, and when the lid housing 120 is closed and the monitor 121 is accommodated in the main body housing 110, the control unit 200 automatically ends the portable diagnostic ultrasound apparatus 100 safely.

Hereinafter, in this description, a state in which the lid housing 120 is closed and the monitor 121 is accommodated in the main body housing 110 is referred to as a state in which the monitor 121 is closed, and a state in which the lid housing 120 is opened from the closed state will be referred to as a state in which the monitor 121 is open. To end the portable diagnostic ultrasound apparatus 100 means to cut power supply to the portable diagnostic ultrasound apparatus 100; that is, to turn off (to power off, to shut off) power of the portable diagnostic ultrasound apparatus 100.

In the portable diagnostic ultrasound apparatus 100, other than transmission/receipt of ultrasonic waves and formation of images, processing of displaying an image, processing of inputting a comment in the displayed image, processing of making measurements of the displayed image, processing of storing formed images, measurement results, and the like in a storage device such as a hard disk and the like provided in the portable diagnostic ultrasound apparatus 100, processing of transferring the image, measurement results, and the like stored in the storage device to an external medium and the like are executed.

Some of these processing operations need time for the processing to be completed. Moreover, data might be broken or an error might occur in some of them if power supply is inadvertently interrupted (power is turned off) in the middle of execution. Based on these situations, the control unit 200 of this embodiment ends the ongoing processing safely and turns off power of the portable diagnostic ultrasound apparatus 100 without corruption of data or occurrence of an error after a predetermined period of time has elapsed since the monitor 121 is closed.

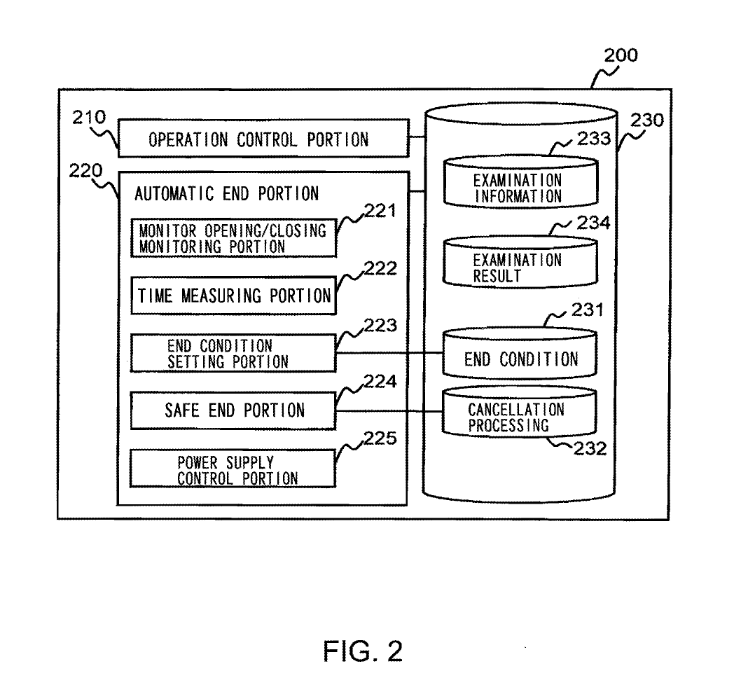

FIG. 2 is a functional block diagram of the control unit 200 of this embodiment. As illustrated in this figure, he control unit 200 of this embodiment is provided with an operation control portion 210 for controlling an operation of each portion, an automatic end portion 220 for executing automatic end processing for automatically ending the portable diagnostic ultrasound apparatus 100 safely after a predetermined period of time has elapsed since the monitor 121 is closed, and the storage portion 230 for storing data required for control of the operation, data required for various types of processing, data obtained as the result of the operation, data obtained as the result of various types of processing, and the like.

The automatic end portion 220 is provided with a monitor opening/closing monitoring portion 221, a time measuring portion 222, an end condition setting portion 223, a safe end portion 224, and a power supply control portion 225. Moreover, the storage portion 230 is provided with an end condition storage portion 231, a cancellation processing storage portion 232, an examination information storage portion 233, and an examination result storage portion 234.

The examination information storage portion 233 stores various types of examination information required for obtaining ultrasonic images, for measurement using the obtained ultrasonic images and the like in the portable diagnostic ultrasound apparatus 100. The examination information includes information relating to the subject 101 (subject information), setting information of the portable diagnostic ultrasound apparatus 100 (apparatus setting information), and the like, for example. The subject information includes patient information of the subject 101, information on a portion to be examined, information by examination type, and the like. The apparatus setting information includes a set value of an image quality parameter and the like.

The examination result storage portion 234 stores the ultrasonic images obtained in the portable diagnostic ultrasound apparatus 100, the measurement results using the obtained ultrasonic images, and the like.

The operation control portion 210 extracts, from among the examination information stored in the examination information storage portion 233, the examination information used for an examination specified by a user to be executed, obtains a diagnostic ultrasound image by controlling each portion of the portable diagnostic ultrasound apparatus 100 by using the extracted examination information, and stores the image in the examination result storage portion 234.

The monitor opening/closing monitoring portion 221 monitors opening/closing of the monitor 121. In this embodiment, the closed signal or the open signal from the sensor provided in the monitor 121 is received.

The time measuring portion 222 starts measurements of elapsed time when the monitor opening/closing monitoring portion 221 receives the closed signal. The measurement of the elapsed time is performed until time set as an end delayed time which will be described later has elapsed or until the open signal is received from the monitor opening/closing monitoring portion 221.

The end condition setting portion 223 receives various settings relating to the automatic end processing from the operator through the operation portion 111 and stores them in the end condition storage portion 231. The end condition stored in the end condition storage portion 231 includes, for example, end delay time T which is time until the power of the entire portable diagnostic ultrasound apparatus 100 is turned off after the monitor 121 is closed, setting at automatic end, or presence of notification at start after automatic end. Regarding the end condition, an initial value may be held in advance.

If the initial value is stored, when no setting is given by the operator, the initial value is used.

The setting at automatic end is a setting whether the ongoing processing at that point of time is stopped and cancelled after the end delay time T has elapsed after the monitor 121 is closed or the end of the execution is to be waited (standby). The setting at automatic end may be the same condition for all the processing operations or may be set for each processing operation. For example, such setting may be made that in the case of processing of performing measurement of the displayed image, completion of the processing is awaited, while in the case of data transfer processing to the external media, the processing is cancelled in the middle. In this case, specification from the operator is received for each processing operation which is likely to be executed in the portable diagnostic ultrasound apparatus 100. Alternatively, the initial value of the setting at automatic end may be registered in advance for each processing operation.

For example, if 30 minutes is set for the end delay time T, and data transfer requiring 1 hour until the data transfer is completed has been started immediately before the monitor 121 is closed, forced turning-off of power of the portable diagnostic ultrasound apparatus 100 at the time when 30 minutes has elapsed since the monitor 121 is closed might cause corruption or loss of data. In order to avoid such a situation, power is turned off after safe end processing such as cancellation processing of data saving or waiting for all the transfers to be completed is executed in this embodiment.

The presence of notification at start after the automatic end is a setting on whether or not to notify the operator that the immediately preceding end is by the automatic end processing by the automatic end portion 220 in this embodiment. If setting is made to notify, the operator can identify whether the immediately preceding end is caused by power off by the operator or the automatic end. In the case of the automatic end, the processing cancelled at the previous end is resumed in this embodiment.

Regarding the end delay time T, an upper limit value may be set in advance if the operator effects setting from the operation portion 111. As a result, the automatic end processing can be reliably executed within a time determined in advance.

The safe end portion 224 executes safety processing for safely ending the ongoing processing and processing waiting to be executed in accordance with the setting at automatic end. More specifically, if the setting at automatic end is to cancel the ongoing processing and the processing waiting to be executed, the processing being executed at that point of time is stopped and cancelled. At this time, information specifying the cancelled processing or the like is stored as cancellation information in the cancellation processing storage portion 232, normal end processing is executed, and end of the processing is notified to the power supply control portion 225. On the other hand, if the setting is to wait for end of all the ongoing processing and processing waiting to be executed, end of all the processing is waited for, normal end processing is executed, and processing end is notified to the power supply control portion 225.

Here, the information to be stored as cancellation information in the cancellation processing storage portion 232 is, for example, information indicating progress states associated with information that can specify the processing, for the ongoing processing and the like. The information indicating progress states includes, more specifically, information indicating the extent to which transfer has been completed, information specifying data not having been transferred, and the like when the data transfer processing is to be cancelled in the middle. Moreover, regarding the processing waiting to be executed, the information to be stored is information indicating the processing associated with information that can specify the processing and not having been executed.

In the cancellation processing of the ongoing processing, the processing is executed up to a processing step that can be stopped, and various types of data used in the processing as necessary are saved similarly to cancellation of general processing.

Moreover, in the normal end processing, applications steadily executed in the portable diagnostic ultrasound apparatus 100; that is, a database engine used for image data management, for example, is stopped, software for driving the diagnostic ultrasound apparatus is ended, and the Operating System (hereinafter referred to as OS) of the processing device is ended.

The power supply control portion 225 controls supply of power from the power supply 112 to each portion in the portable diagnostic ultrasound apparatus 100 from the power supply 112. If the monitor opening/closing monitoring portion 221 receives a closed signal, for example, supply of power to the ultrasonic transmission/receipt unit 113 is stopped, and output of the ultrasonic waves is stopped. Moreover, if the monitor opening/closing monitoring portion 221 similarly receives a closed signal, supply of power to the monitor 121 and the operation portion 111 is stopped. Furthermore, if notification of processing end is received from the safe end portion 224, power of the entire portable diagnostic ultrasound apparatus 100 is turned off.

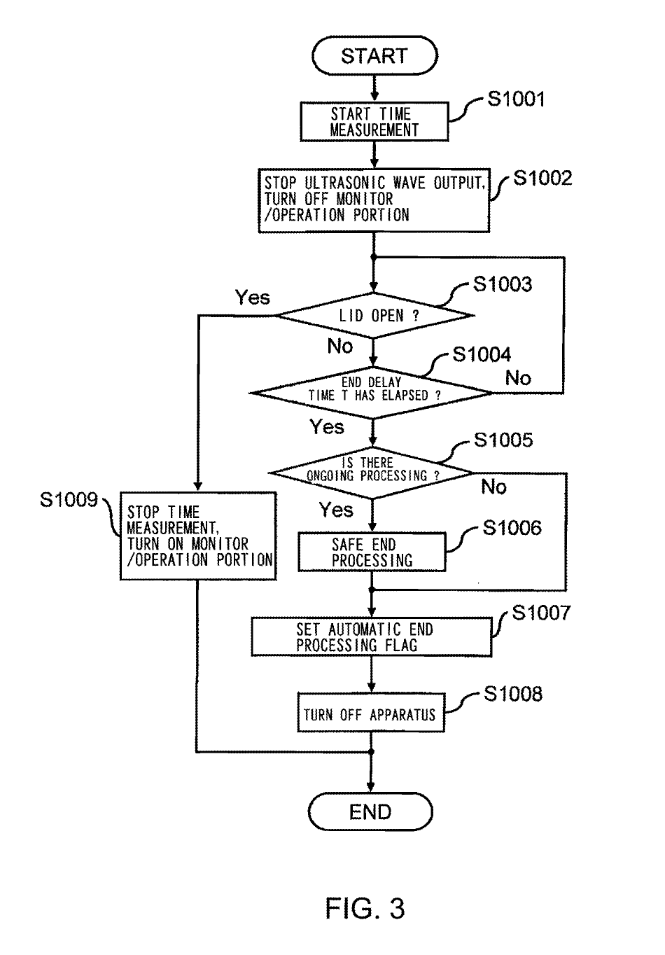

Subsequently, a flow of the automatic end processing by the automatic end portion 220 of this embodiment will be explained. FIG. 3 shows a processing flow of the automatic end processing of this embodiment. The automatic end processing of this embodiment is started by using as a trigger receipt of a closed signal from the monitor 121 by the monitor opening/closing monitoring portion 221. Moreover, the various end conditions are set through the end condition setting portion 223 in advance and held in the end condition storage portion 231. Initial values may be held in the end condition storage portion 231 in advance.

When the monitor opening/closing monitoring portion 221 receives the closed signal, the time measuring portion 222 starts time measurement (Step S1001). Then, the power supply control portion 225 stops supply of power to the ultrasonic transmission/receipt unit 113, the monitor 121, and the operation portion 111, stops output of ultrasonic waves, and turns off the power supply of the monitor 121 and the operation portion 111 (Step S1002). As a result, video output of the monitor 121 and LED of the operation portion 111 are turned off, and power consumption of the portable diagnostic ultrasound apparatus 100 is reduced. Moreover, transmission/receipt of ultrasonic waves from the ultrasonic probe 130 is stopped, and ultrasonic images are brought into a freeze state.

After that, the automatic end portion 220 waits for elapse of the end delay time T stored in the end condition storage portion 231 (Step S1004). If the lid housing 120 is opened, and the monitor opening/closing monitoring portion 221 receives an open signal during this period (Step S1003), the automatic end portion 220 performs the time measuring portion 222 stop time measurement, makes the power supply control portion 225 resume supply of power from the power supply 112 to the monitor 121 and the operation portion 111 (Step S1009), and ends the automatic end processing.

If the lid housing 120 is opened during the end delay time T, supply of power to the ultrasonic transmission/receipt unit 113 is not resumed. Therefore, the video output of the monitor 121 and the LED of the operation portion 111 are tuned on, while the freeze state of the ultrasonic image is maintained. Cancellation of stop of transmission/receipt of ultrasonic waves in the ultrasonic transmission/receipt unit 113 is performed manually through the operation portion 111.

If the end delay time T has elapsed without receiving the open signal, the automatic end portion 220 discriminates whether or not there is ongoing processing and/or processing waiting to be executed (Step S1005). If it is discriminated to be Yes, the safe end portion 224 is made to execute safe end processing (Step S1006). The fact that the immediately preceding end is the automatic end processing is stored in the cancellation processing storage portion 232 (Step S1007). Here, an automatic end flag or the like is set, for example. Then, the power supply control portion 225 is made to turn off the entire portable diagnostic ultrasound apparatus 100 (Step S1008), and the automatic end processing is ended. If it is discriminated to be No in Step S1005, the routine proceeds to processing in Step S1007 without executing the safe end processing in Step S1006.

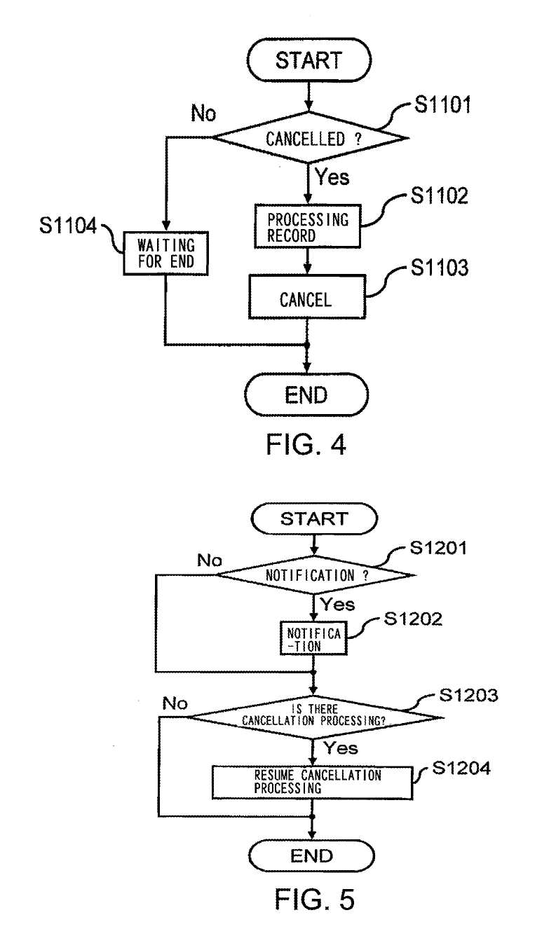

Subsequently, the safe end processing by the safe end portion 224 at Step S1006 will be explained. FIG. 4 shows a processing flow of the safe end processing of this embodiment. As described above, this processing is executed if there is ongoing processing or processing waiting to be executed after the end delay time T has elapsed.

The safe processing portion refers to the end condition storage portion 231 and discriminates whether or not the setting at automatic end is cancellation or standby (Step S1101).

If it is cancellation, the ongoing processing is stopped, and the above-described cancellation information is stored in the cancellation processing storage portion 232 (Step S1102). Then, the stored processing which was being executed and the processing waiting to be executed are cancelled (Step S1103), and the processing is ended.

On the other hand, if the setting at automatic end is standby, completion of execution of the ongoing processing and the processing waiting to be executed is awaited (Step S1104). Then, processing is ended.

If the setting at automatic end is standby, it may be so constituted that only the ongoing processing is completed, while the processing waiting to be executed is cancelled. In this case, in Step S1104, the information specifying the processing waiting to be executed for which execution has been canceled is stored in the cancellation processing storage portion 232 as the cancellation information.

The setting at automatic end may be made different for each of the processing operations. If different settings at end are made for each of the processing operations, the safe end processing by the safe end portion 224 is executed as follows.

For all the ongoing processing operations and processing operations waiting to be executed, their settings are determined. The processing with cancellation setting is cancelled, and the cancellation information is stored in the cancellation processing storage portion 232. On the other hand, the processing waiting to be executed is executed sequentially. When all the processing set to standby has been ended, the safe end processing is ended.

Subsequently, processing at startup of the portable diagnostic ultrasound apparatus 100 of this embodiment (start processing after automatic end) when the immediately preceding end is the above-described automatic end will be explained. FIG. 5 shows a processing flow of the start processing after automatic end of this embodiment. The control unit 200 discriminates whether or not the immediately preceding end is the automatic end by presence of the automatic end processing flag in the cancellation processing storage portion 232. That is, the control unit 200 refers to the cancellation processing storage portion 232 immediately after the start, and if the automatic end processing flag is set, the start processing after automatic end is executed. The control unit 200 removes the setting of the automatic end flag immediately after start of the start processing after automatic end.

The control unit 200 refers to the end condition setting portion 223 and discriminates whether or not to notify that it is automatic end (Step S1201). If it is to be notified, a message indicating that the immediately preceding end is automatic end is displayed on the monitor 121 (Step S1202). The message is created in advance and held in the storage portion 230. If there is no setting of notification, the routine proceeds to Step S1203 as is.

Then, the control unit 200 refers to the cancellation processing storage portion 232 and discriminates whether or not there is processing cancelled in the immediately preceding automatic end (Step S1203). If there is the cancelled processing, the processing is resumed at the stopped spot (Step S1204).

Then, the start processing after automatic end is ended. If there is no cancelled processing in Step S1203, the start processing after automatic end is ended as is.

Here, whether or not to execute the start processing after automatic end is discriminated on the basis of presence of the automatic end processing flag, but this is not limiting. Discrimination may be made on the basis of presence of cancelled processing, for example. In this case, the control unit 200 refers to the cancellation processing storage portion 232 at start and if there is cancelled processing, and the control unit 200 executes the start processing after automatic end. In this case, the processing of setting the automatic end flag is not necessary.

Moreover, if there is cancelled processing in the start processing after automatic end, it may be so configured that the cancelled processing is displayed on the monitor 121 and selection on whether or not to resume the processing or whether or not to execute the processing is received from the user. The presentation is performed by displaying the information specifying the cancelled processing on the monitor 121 or the like.

As described above, the portable diagnostic ultrasound apparatus 100 of this embodiment is the portable diagnostic ultrasound apparatus 100 provided with the main body housing 110 including the ultrasonic transmission/receipt unit 113 for generating and transmitting the ultrasonic waves to the ultrasonic probe and for receiving the ultrasonic waves from the ultrasonic probe, the ultrasonic image forming unit 114 for forming the ultrasonic image on the basis of the ultrasonic waves received in the ultrasonic transmission/receipt unit 113, and the control unit 200 and the lid housing 120 including the display device (monitor 121) for displaying the ultrasonic image, and the lid housing 120 is attached to the main body housing 110, capable of being opened/closed, and the control unit 200 is provided with the automatic end portion 220 for ending the diagnostic ultrasound apparatus 100 (shutting off supply of power to the portable diagnostic ultrasound apparatus 100) when the lid housing 120 is closed and the safe end portion 224 for executing safe end processing for safely ending the ongoing processing and processing scheduled to be executed in the portable diagnostic ultrasound apparatus 100.

The safe end portion 224 may execute the safe end processing after the end delay time which is time determined in advance has elapsed since the lid housing 120 and the main body housing 110 are closed. Moreover, the safe end portion 224 may cancel the ongoing processing and the processing scheduled to be executed and hold the progress state at cancellation in association with the cancelled processing.

Moreover, if the previous end is the end by the automatic end portion, at startup of the portable diagnostic ultrasound apparatus, the control unit 200 may resume the processing held in association with the progress state from the state at cancellation.

Moreover, the automatic end portion 220 may be so configured that supply of power to the ultrasonic transmission/receipt unit 113 and the display device (monitor 121) is shut off, when the lid housing 120 is closed. Moreover, the automatic end portion 220 may be so configured that, when the lid housing 120 is opened during the end delay time, supply of power to the display device (monitor 121) is resumed.

Furthermore, the safe end portion 224 may be so configured that, for the ongoing processing, end of processing is to be awaited in accordance with the user's setting.

As described above, according to this embodiment, opening/closing of the monitor 121 is monitored, and after the predetermined time has elapsed since the monitor 121 is closed, the ongoing processing and the processing waiting to be executed are safely cancelled, and the power of the portable diagnostic ultrasound apparatus 100 is turned off. Therefore, according to this embodiment, even if the monitor 121 is closed, and the video image outputted onto the monitor 121 cannot be checked, the portable diagnostic ultrasound apparatus 100 is not operated for a long time. Thus, deterioration of hardware by long-term electric conduction can be prevented. Moreover, since the ongoing processing is safely ended at the automatic end, corruption or loss of data can be prevented. That is, since the diagnostic ultrasound apparatus is used only in a range for which verification has been reliably made, safety in use of the diagnostic ultrasound apparatus can be improved.

Moreover, if the immediately preceding end is automatic end, the cancelled processing is resumed at the start next time. Therefore, the processing is not left as is after being cancelled but is executed. Therefore, according to this embodiment, power can be safely and appropriately turned off when the portable diagnostic ultrasound apparatus 100 is not in use, without discarding the processing intended by the user.

In the above-described embodiment, the timing of the automatic end is after the set predetermined time (end delay time T) has elapsed, but this is not limiting. For example, the timing may be when the ongoing processing is all ended. In this case, the time measuring portion 222 is not necessary. Moreover, in the above-described flow of the automatic end processing, the processing of discriminating elapse of the set time in Step S1004 is not necessary, either. Instead, a processing end discriminating portion for discriminating end of execution of the ongoing processing is provided. Then, if it is discriminated in Step S1005 that there is ongoing processing, the processing end discriminating portion monitors progress of the processing and after the end of the processing, turns off the apparatus power. During this progress monitoring, the safe end portion 224 is made to store the processing waiting to be executed and to safely cancel it.

### Second Embodiment

Subsequently, a second embodiment to which the present invention is applied will be explained. In this embodiment, when the automatic end is executed, examination information at the automatic end is stored in addition to the information of ongoing processing and/or processing waiting to be executed.

A configuration of the portable diagnostic ultrasound apparatus 100 and a functional block of the control unit 200 of this embodiment are basically similar to those of the first embodiment. However, in this embodiment, the information to be stored at the automatic end is different from that in the first embodiment as described above. Therefore, the processing of the automatic end portion 220, the end condition stored in the end condition storage portion 231, and the information stored in the cancellation processing storage portion 232 are different. This embodiment will be explained below by focusing on the features of configuration different from those of the first embodiment.

The automatic end portion 220 of this embodiment stores the examination information of the examination being conducted at the automatic end or the examination being conducted immediately before the automatic end after the safe end processing or after the end delay time T has elapsed in the cancellation processing storage portion 232. The examination information to be stored is assumed to be relating to the examination being conducted in the portable diagnostic ultrasound apparatus 100 when the monitor 121 is closed or immediately before as described above. Therefore, in this embodiment, the above-described examination information is stored in addition to the cancellation information stored in the first embodiment in the cancellation processing storage portion 232.

Moreover, in the end condition storage portion 231, there is stored information on whether to store the examination information in addition to the end condition stored in the first embodiment, whether or not to resume the examination under the same condition as that at the automatic end in the start processing after the automatic end, whether or not to display a dialog for confirming an intension of resumption, or the like.

Regarding these end conditions, too, the condition inputted by the operator through the operation portion 111 is stored by the end condition setting portion 223 in the end condition storage portion 231. An initial value may be set in advance in the end condition storage portion 231. In this case, if no input is made by the operator, the initial value is used, whereas if an input is made by the operator, the inputted latest condition is used.

As the end condition, regarding presence of storage of the examination information, whether or not to store the examination information may be set for each type of the information. In this embodiment, it is assumed below that presence of storage of subject information and presence of apparatus setting information are set separately.

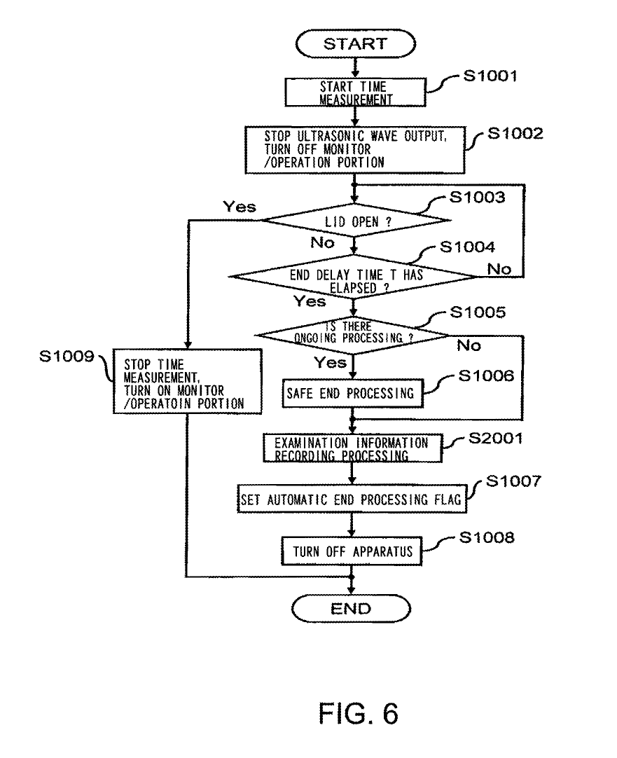

A flow of the automatic end processing by the automatic end portion 220 in this embodiment will be explained. FIG. 6 shows a processing flow of the automatic end processing of this embodiment. The automatic end processing of this embodiment is started by using as a trigger receipt of the closed signal from the monitor 121 by the monitor opening/closing monitoring portion 221. Moreover, various end conditions are stored in advance in the end condition storage portion 231.

The automatic end processing of this embodiment is basically similar to that of the automatic end processing of the first embodiment explained by reference to FIG. 3. That is, when the monitor opening/closing monitoring portion 221 receives the closed signal, the time measuring portion 222 starts time measurement (Step S1001). Then, the power supply control portion 225 stops supply of power to the ultrasonic transmission/receipt unit 113, the monitor 121, and the operation portion 111, stops output of ultrasonic waves, and turns off the power supply of the monitor and the operation portion (Step S1002). After that, the automatic end portion 220 waits for elapse of the end delay time T set by the end condition setting portion 223 (Step S1004). During this period, when the monitor opening/closing monitoring portion 221 receives the open signal, the automatic end portion 220 determines that the lid housing 120 has been opened (Step S1003), makes the time measuring portion 222 stop time measurement, makes the power supply control portion 225 resume supply of power from the power supply 112 to the monitor 121 and the operation portion 111 (Step S1009), and ends the automatic end processing.

If the end delay time T has elapsed without receiving the open signal, the automatic end portion 220 discriminates whether or not there is ongoing processing including the processing waiting to be executed (Step S1005). If it is discriminated to be yes, the safe end portion 224 is made to execute the safe end processing (Step S1006). Then, the fact that the immediately preceding end is the automatic end processing is stored in the cancellation processing storage portion 232 (Step S1007). Then, the power supply control portion 225 is made to turn off the entire portable diagnostic ultrasound apparatus 100 (Step S1008), and the automatic end processing is ended.

However, in this embodiment, the automatic end portion 220 executes examination information storage processing for storing the examination information in the cancellation processing storage portion 232 immediately before Step S1007 (Step S2001). That is, after the safe end processing in Step S1006 or if it is determined that there is no ongoing processing in Step S1005, the examination information storage processing is executed after Step S1005 and Step 1006.

The safe end processing by the safe end portion 224 in Step S1006 is similar to that of the safe end processing in the first embodiment explained by reference to FIG. 4.

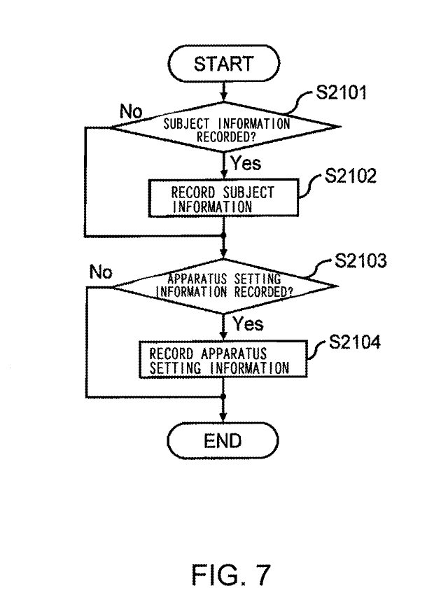

Subsequently, the examination information storage processing by the automatic end portion 220 of this embodiment will be explained. FIG. 7 shows a processing flow of the examination information storage processing of this embodiment.

The automatic end portion 220 refers to the end condition storage portion 231 and discriminates whether or not subject information is set to be stored as the end condition (Step S2101). If it is set to be stored, the subject information used for the immediately preceding examination in the subject information stored in the examination information storage portion 233 is stored in the cancellation processing storage portion 232 (Step S2102), and the routine proceeds to the subsequent Step. If it is not set, the routine proceeds to Step S2103 as is.

The automatic end portion 220 refers to the end condition storage portion 231 and discriminates whether or not apparatus setting information is set to be stored as the end condition (Step S2103). If it is set to be stored, the subject information used for the immediately preceding examination in apparatus setting information stored in the examination information storage portion 233 is stored in the cancellation processing storage portion 232 (Step S2104), and the processing is ended. If it is not set, the processing is ended as is.

Here, discrimination on presence of storage of the examination information and storage processing are executed in order of the subject information and the apparatus setting information, but the processing order is not limited to that.

Subsequently, the start processing after the automatic end by the control unit 200 of this embodiment will be explained. FIG. 8 shows a processing flow of the start processing after the automatic end of this embodiment. The start processing after the automatic end of this embodiment is basically similar to that of the start processing after the automatic end of the first embodiment explained by reference to FIG. 5. That is, the fact that the immediately preceding processing is the automatic end is notified in accordance with the setting, and if there is cancelled processing, it is resumed (Steps S1201 to S1204).

After that, in this embodiment, it is determined whether or not the examination is to be resumed under the same condition as that in the automatic end processing. The subsequent processing is as follows. First, the control unit 200 refers to the end condition storage portion 231 and discriminates whether or not the setting is inquiring an intention of restoration (Step S2201).

If the intention is inquired about in setting, a confirmation dialogue is displayed on the monitor 121, and an instruction from the operator is received (Step S2202). Data used for the display of the conformation dialogue is held in the storage portion in advance. If setting does not inquire the intention, the routine proceeds to Step S2203 as is.

Then, if the operator gives an instruction of restoration, or if it is set to restore at time of resumption in the end condition storage portion 231 regardless of the intention of the operator (Step S2203), the control unit 200 restores the examination information stored in the cancellation processing storage portion 232 (Step S2204) and ends the processing. On the other hand, in the other cases, the examination information stored in the cancellation processing storage portion 232 is discarded (Step S2205), and the processing is ended.

The above-described processing in Steps S1201 to S1204 does not have to be executed.

As described above, the portable diagnostic ultrasound apparatus 100 of this embodiment is the portable diagnostic ultrasound apparatus 100 provided with the main body housing 110 including the ultrasonic transmission/receipt unit 113 for generating and transmitting the ultrasonic waves to the ultrasonic probe and receiving the ultrasonic waves from the ultrasonic probe, the ultrasonic image forming unit 114 for forming the ultrasonic image on the basis of the ultrasonic waves received in the ultrasonic transmission/receipt unit 113, and the control unit 200 and the lid housing 120 including the display device (monitor 121) for displaying the ultrasonic image, and the lid housing 120 is attached to the main body housing 110, capable of being opened/closed, and the control unit 200 is provided with the automatic end portion 220 for ending the diagnostic ultrasound apparatus 100 (shutting off supply of power to the portable diagnostic ultrasound apparatus 100) when the lid housing 120 is closed and the safe end portion 224 for executing the safe end processing for safely ending the ongoing processing and the processing scheduled to be executed in the portable diagnostic ultrasound apparatus 100.

The safe end portion 224 may execute the safe end processing after the end delay time which is the time determined in advance has elapsed since the lid housing 120 and the main body housing 110 are closed. Moreover, the safe end portion 224 may cancel the ongoing processing and the processing scheduled to be executed and hold the progress state at cancellation in association with the cancelled processing.

Moreover, if the previous end is the end by the automatic end portion, at startup of the portable diagnostic ultrasound apparatus, the control unit 200 may resume the processing held in association with the progress state from the state at cancellation.

Moreover, the automatic end portion 220 may store the examination information of the examination conducted in the portable diagnostic ultrasound apparatus immediately before the lid housing 120 is closed. The control unit 200 may restore the examination information stored by the automatic end portion 220 at startup of the portable diagnostic ultrasound apparatus and conduct the examination in accordance with the restored examination information.

Moreover, the automatic end portion 220 may be so configured that, when the lid housing 120 is closed, supply of power to the ultrasonic transmission/receipt unit 113 and the display device (monitor 121) is shut off. Moreover, the automatic end portion 220 may be so configured that, when the lid housing 120 is opened during the end delay time, supply of power to the display device (monitor 121) is resumed.

Furthermore, the safe end portion 224 may be so configured that, for the ongoing processing, end of processing is to be awaited in accordance with the user's setting.

Thus, according to this embodiment, deterioration of hardware caused by continuous electric conduction can be suppressed without increasing user workload. Thus, safety of the apparatus is improved.

Moreover, in this embodiment, information indicating a state of the examination conducted at that time; that is, the name of the subject, a date of birth of the subject, or the like is stored at an arbitrary timing. As described above, according to this embodiment, the examination information of the examination being conducted immediately before the automatic end is stored and the examination information can be restored at the subsequent startup, and thus, the examination can be resumed under the same condition as that before the automatic end. There is no need to make setting again when the examination is continued, whereby the operator workload can be reduced, and occurrence of erroneous examination can be suppressed at the same time.

### Third Embodiment

Subsequently, a third embodiment to which the present invention is applied will be explained. In this embodiment, the subsequent automatic end processing is changed in accordance with the direction when the monitor 121 is closed.

In general, the monitor 121 (lid housing 120) is used in a state opened perpendicularly or substantially perpendicularly to the main body housing 110 of the portable diagnostic ultrasound apparatus 100. The main body housing 110 is generally arranged and used horizontally. At this time, if the monitor 121 (lid housing 120) is closed to the main body housing 110 side, it often occurs when the operator temporarily suspends use. On the other hand, if the monitor 121 (lid housing 120) is closed and then, brought into a vertical state, in many cases the user intends to carry the portable diagnostic ultrasound apparatus 100.

In this embodiment, paying attention to this feature, after detection that the monitor 121 has been closed, the direction of the monitor 121 (lid housing 120) is detected, and if it is horizontal, ongoing processing is safely ended after a predetermined time has elapsed, and power of the apparatus is turned off. On the other hand, if it is vertical, the ongoing processing is safely ended immediately after that, and power of the apparatus is turned off.

The portable diagnostic ultrasound apparatus 100 of this embodiment which realizes that will be explained. The portable diagnostic ultrasound apparatus 100 of this embodiment basically has a configuration similar to that of the first embodiment.

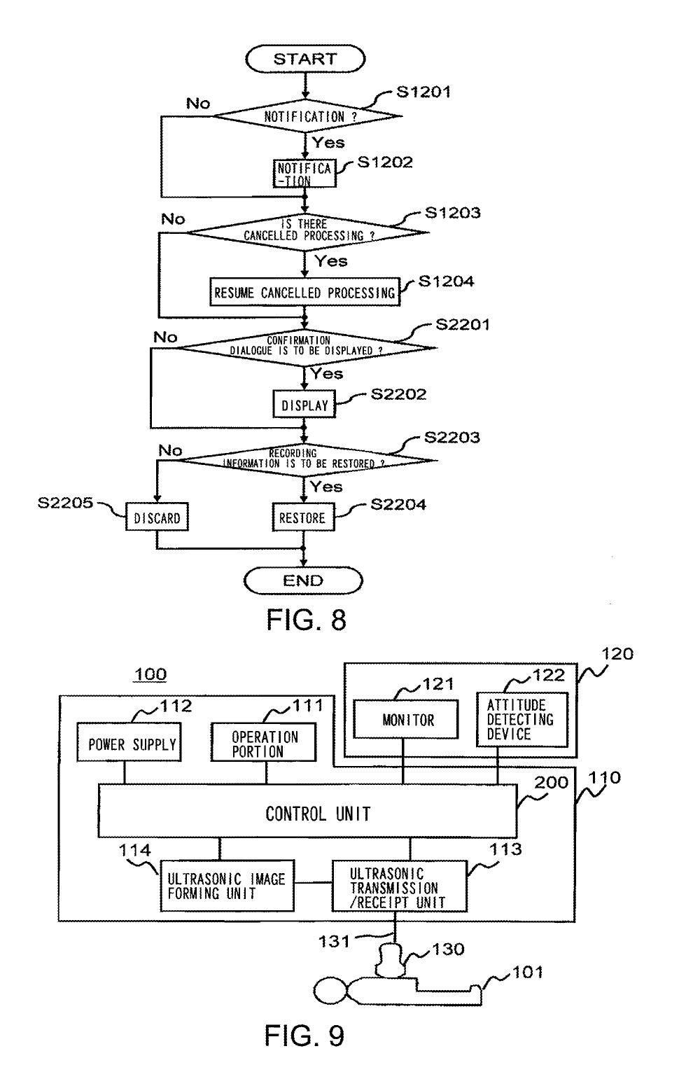

However, as illustrated in FIG. 9, the portable diagnostic ultrasound apparatus 100 of this embodiment is provided with an attitude detecting device 122 for detecting an attitude (direction) of the lid housing 120 (monitor 121). Here, an instance that the attitude detecting device 122 is arranged in the lid housing 120 is exemplified.

For the attitude detecting device 122, a gyro or the like, for example, is used. The attitude detecting device 122 outputs a signal indicating the direction (attitude) of the lid housing 120 (monitor 121) at that point of time to the control unit 200 at predetermined time intervals. Here, a signal outputted when the direction of the lid housing 120 (monitor 121) is horizontal is referred to as a horizontal signal, and a signal outputted in the case of the vertical is referred to as a vertical signal.

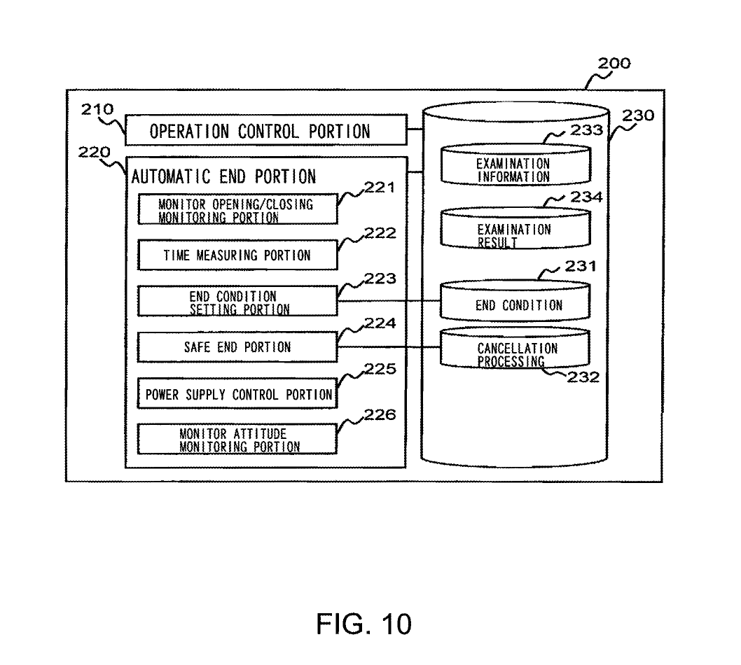

A functional block of the control unit 200 of this embodiment is illustrated in FIG. 10. As illustrated in this figure, the control unit 200 of this embodiment is further provided with a monitor attitude monitoring portion 226 for monitoring an attitude of the lid housing 120 (monitor 121) in addition to the configuration of the first embodiment illustrated in FIG. 2.

The monitor attitude monitoring portion 226 receives the horizontal signal or the vertical signal from the attitude detecting device 122 and discriminates the attitude of the monitor 121. In this embodiment, if the monitor attitude monitoring portion 226 receives the horizontal signal after a predetermined time Δt has elapsed since the monitor opening/closing monitoring portion 221 receives the closed signal, the monitor attitude monitoring portion 226 instructs the automatic end portion 220 to execute the safe end processing after waiting only for the end delay time T, similarly to each of the above embodiments. On the other hand, if the vertical signal is received, it instructs execution of the safe end processing without waiting for elapse of the end delay time T.

The automatic end portion 220 of this embodiment discriminates presence of execution processing in accordance with an instruction from the monitor attitude monitoring portion 226 and then, makes the safe end portion 224 execute the safe end processing as necessary.

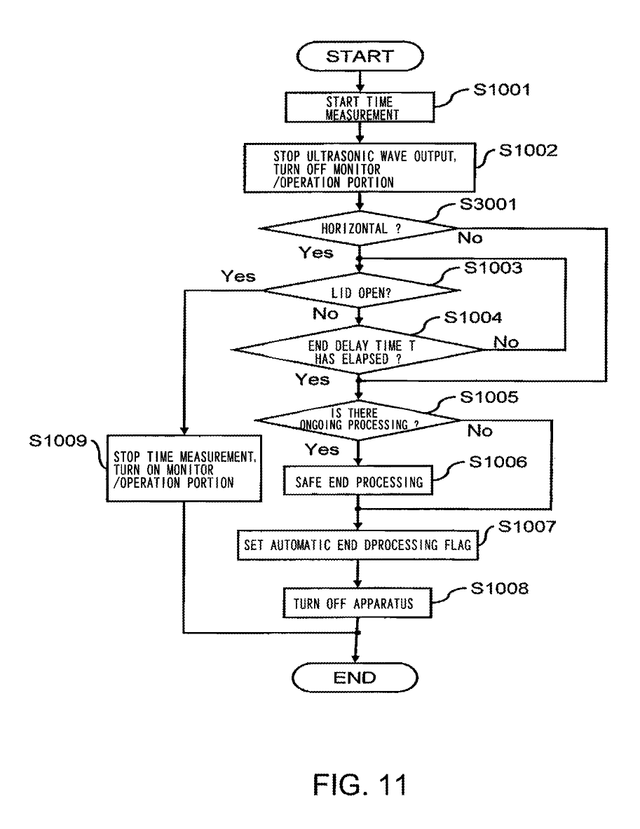

A flow of the automatic end processing by the automatic end portion 220 of this embodiment will be described. FIG. 11 shows a processing flow of the automatic end processing of this embodiment. The automatic end processing of this embodiment is started while using as a trigger receipt of the closed signal from the monitor 121 by the monitor opening/closing monitoring portion. Moreover, various end conditions are stored in the end condition storage portion 231 in advance.

The automatic end processing of this embodiment is basically similar to that of the automatic end processing of the first embodiment explained by reference to FIG. 3. That is, when the monitor opening/closing monitoring portion 221 receives the closed signal, the time measuring portion 222 starts time measurement (Step S1001). Then, the power supply control portion 225 stops supply of power to the ultrasonic transmission/receipt unit 113, the monitor 121, and the operation portion 111, stops output of ultrasonic waves, and turns off the power supply of the monitor and the operation portion (Step S1002).

After that, in this embodiment, the monitor attitude monitoring portion 226 discriminates whether or not the monitor 121 is horizontal (Step S3001). Here, discrimination is made on the basis of whether or not the horizontal signal has been received. If the horizontal signal has been received, the automatic end portion 220 waits only for the end delay time T (Step S1004), similarly to the automatic end processing of the first embodiment, and if there is ongoing processing (Step S1005), executes the safe end processing (Step S1006), sets the automatic end processing flag (Step S1007), turns off power of the apparatus (Step S1008), and ends the processing. If the monitor 121 is opened during standby for the end delay time T, too, time measurement is stopped, power of the monitor 121 and the operation portion 111 is turned off, and the processing is ended, similarly to the first embodiment.

On the other hand, if the horizontal signal has not been received in Step S3001, the automatic end portion 220 proceeds to Step S1005 without waiting for the end delay time T.

The safe end processing by the safe end portion 224 of this embodiment is similar to that of the first embodiment. Moreover, the start processing after the automatic end is similar to that of the first embodiment.

Here, execution of the processing in the first embodiment is explained as an example of the automatic end processing executed in this embodiment, but this is not limiting. It may be the processing of the second embodiment. In this case, the processing at the start immediately after the automatic end is assumed to be also similar to that of the second embodiment.

As described above, the portable diagnostic ultrasound apparatus 100 of this embodiment is the portable diagnostic ultrasound apparatus 100 provided with the main body housing 110 including the ultrasonic transmission/receipt unit 113 for generating and transmitting the ultrasonic waves to the ultrasonic probe and receiving the ultrasonic waves from the ultrasonic probe, the ultrasonic image forming unit 114 for forming the ultrasonic image on the basis of the ultrasonic waves received in the ultrasonic transmission/receipt unit 113, and the control unit 200 and the lid housing 120 including the display device (monitor 121) for displaying the ultrasonic image, and the lid housing 120 is attached to the main body housing 110, capable of being opened/closed, and the control unit 200 is provided with the automatic end portion 220 for ending the diagnostic ultrasound apparatus 100 (shutting off supply of power to the portable diagnostic ultrasound apparatus 100) when the lid housing 120 is closed and the safe end portion 224 for executing safe end processing for safely ending the ongoing processing and processing scheduled to be executed in the portable diagnostic ultrasound apparatus 100.

The safe end portion 224 may execute the safe end processing after the end delay time which is the time determined in advance has elapsed since the lid housing 120 and the main body housing 110 are closed. Moreover, the safe end portion 224 may cancel the ongoing processing and the processing scheduled to be executed and hold the progress state at cancellation in association with the cancelled processing.

Moreover, if the previous end is the end by the automatic end portion, at startup of the portable diagnostic ultrasound apparatus, the control unit 200 may resume the processing held in association with the progress state from the state at cancellation.

Moreover, the automatic end portion 220 may store the examination information on the examination conducted in the portable diagnostic ultrasound apparatus immediately before the lid housing 120 is closed. The control unit 200 may restore the examination information stored by the automatic end portion 220 at startup of the portable diagnostic ultrasound apparatus and conduct the examination in accordance with the restored examination information.

Moreover, the attitude detecting device 122 for detecting the attitude of the lid housing 120 after the lid housing 120 is closed may be further provided, and if it is detected that the attitude of the lid housing 120 is other than horizontal, the safe end portion 224 may execute the safe end processing immediately after the detection without waiting for elapse of the end delay time.

Moreover, the automatic end portion 220 may be so configured that, when the lid housing 120 is closed, supply of power to the ultrasonic transmission/receipt unit 113 and the display device (monitor 121) is shut off. Moreover, the automatic end portion 220 may be so configured that, when the lid housing 120 is opened during the end delay time, supply of power to the display device (monitor 121) is resumed.

Furthermore, the safe end portion 224 may be so configured that, for the ongoing processing, end of processing is to be awaited in accordance with the user's setting.

Thus, according to this embodiment, deterioration of hardware caused by continuous electric conduction can be suppressed without increasing user workload, as in the first embodiment or the second embodiment. Thus, safety of the apparatus is improved.

Moreover, according to this embodiment, the procedure of the automatic end processing is changed in accordance with the orientation of the portable diagnostic ultrasound apparatus 100 after the monitor 121 (lid housing 120) is closed. That is, in the case of closing so that the monitor 121 (lid housing 120) is directed horizontally, which is considered to be temporary interruption of the use, the safe end processing is executed after the end delay time T, and the procedure is ended. Alternatively, in the case of closing so that the monitor 121 (lid housing 120) is directed perpendicularly, which is considered to be interruption of the use for movement, the safe end processing is executed without waiting for the end delay time T, and the procedure is ended. Therefore, the automatic end processing can be realized appropriately in a mode according to the user's intention.

### Fourth Embodiment

Subsequently, a fourth embodiment to which the present invention is applied will be explained. In this embodiment, at the automatic end, the fact that the automatic end will be executed is notified to the user.

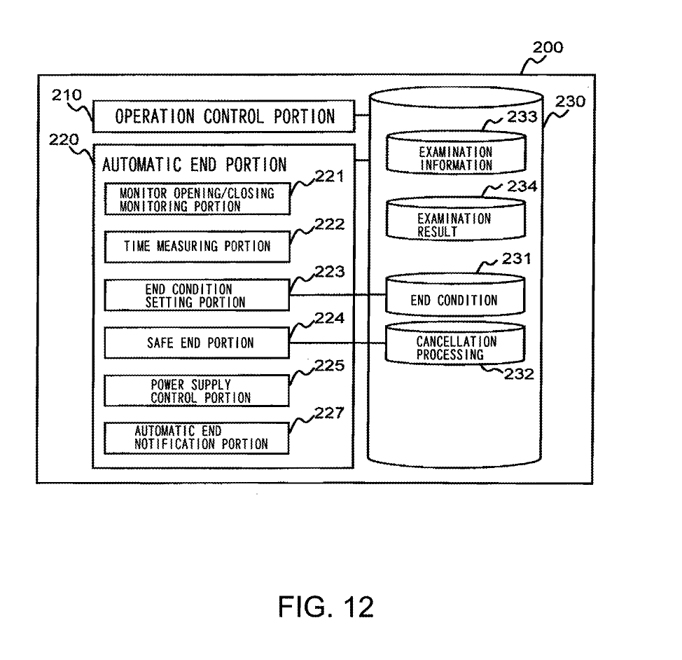

A configuration of the portable diagnostic ultrasound apparatus 100 of this embodiment is basically similar to that of the first embodiment. However, since this embodiment has a notification function as described above, the configuration of the control unit 200 is different. The functional block of the control unit 200 of this embodiment is illustrated in FIG. 12. As illustrated in this figure, the control unit 200 of this embodiment is provided with an automatic end notification portion 227 in the automatic end portion 220 in addition to the configuration of the first embodiment illustrated in FIG. 2.

Moreover, the time measuring portion 222 of this embodiment notifies a measured time to the automatic end notification portion 227 at a predetermined time interval ΔT. Here, the time interval ΔT is assumed to be drastically shorter than the delay end time T. When the automatic end notification portion 227 receives a notice from the time measuring portion 222, the automatic end notification portion 227 sends a notice indicating that the automatic end processing is underway. The notice is made such that, for example, a notice is given to a sub monitor provided in the portable diagnostic ultrasound apparatus 100, an LED provided in the portable diagnostic ultrasound apparatus 100 is flashed, a beep sound is outputted from a speaker provided in the portable diagnostic ultrasound apparatus 100, or the like.

The automatic end notification portion 227 may be so configured that time until the automatic end is calculated and is notified to the operator. Time LT until the automatic end is calculated by LT=T−m×ΔT, assuming that the end delay time is T, and the number of times of receipts of ΔT is m. In this case, the calculated time LT is displayed on the sub monitor at every ΔT.

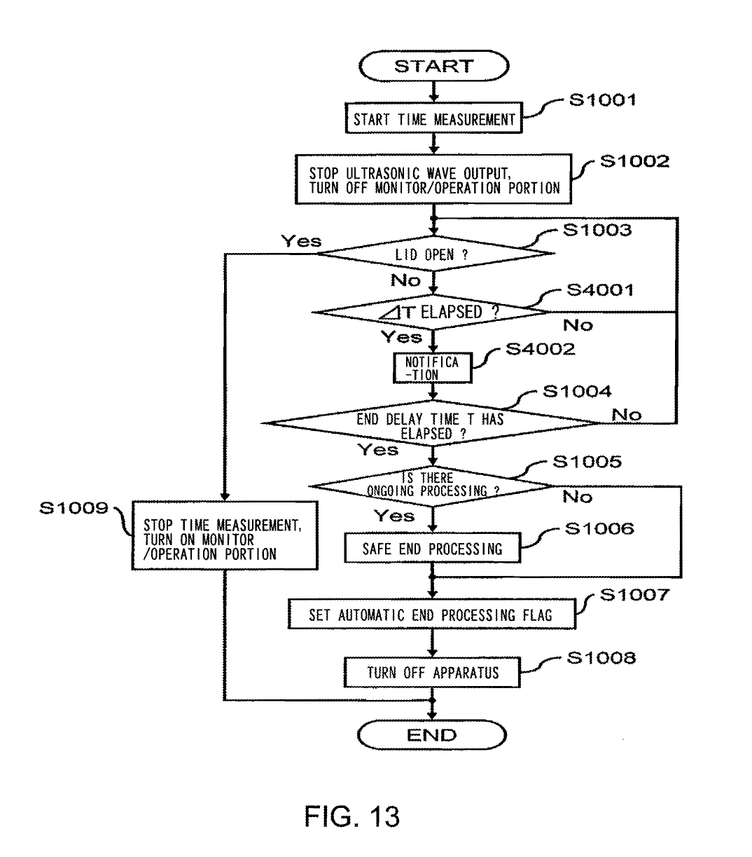

A flow of the automatic end processing by the automatic end portion 220 of this embodiment will be explained by reference to FIG. 13. The automatic end processing of this embodiment is basically similar to any one of the above described embodiments. Here, an instance basically similar to the first embodiment will be explained as an example.

Similarly to the first embodiment, when the monitor opening/closing monitoring portion 221 receives the closed signal, the time measuring portion 222 starts time measurement (Step S1001). Then, the power supply control portion 225 stops supply of power to the ultrasonic transmission/receipt unit 113, the monitor 121, and the operation portion 111, stops output of ultrasonic waves, and turns off the power supply of the monitor and the operation portion (Step S1002). After that, the automatic end portion 220 waits for elapse of the end delay time T set by the end condition setting portion 223 (Step S1004). During this period, when the monitor opening/closing monitoring portion 221 receives the open signal, the automatic end portion 220 determines that the lid housing 120 has been opened (Step S1003), makes the time measuring portion 222 stop time measurement, makes the power supply control portion 225 resume supply of power from the power supply 112 to the monitor 121 and the operation portion 111 (Step S1009), and ends the automatic end processing.

Moreover, in this embodiment, during this period; that is, for a period from start of time measurement after receipt of the closed signal until the end delay time T has elapsed, the automatic end notification portion 227 sends a notice (Step S4002) indicating that the automatic end processing is underway at every ΔT (Step S4001).

The processing after the end delay time T has elapsed without receiving the open signal is similar to that of the first embodiment. That is, the automatic end portion 220 discriminates whether or not there is ongoing processing, including processing waiting to be executed (Step S1005). If it is discriminated to be Yes, the safe end portion 224 is made to execute the safe end processing (Step S1006). The fact that the immediately preceding end is the automatic end processing is stored in the cancellation processing storage portion 232 (Step S1007). Then, the power supply control portion 225 is made to turn off the entire portable diagnostic ultrasound apparatus 100 (Step S1008), and the automatic end processing is ended.

The safe end processing by the safe end portion 224 at Step S1006 is similar to that of the first embodiment. Moreover, the start processing after the automatic end is also similar to that of the first embodiment.

Moreover, in this embodiment, it may be so configured that whether or not to notify the automatic end can be set by the user as the end condition.

Moreover, the automatic end processing which is the basis of this embodiment may be the automatic end processing of the second embodiment or the third embodiment. In this case, the start processing after the automatic end is also made similar to that of the applicable embodiment.

As described above, the portable diagnostic ultrasound apparatus 100 of this embodiment is the portable diagnostic ultrasound apparatus 100 provided with the main body housing 110 including the ultrasonic transmission/receipt unit 113 for generating and transmitting the ultrasonic waves to the ultrasonic probe and receiving the ultrasonic waves from the ultrasonic probe, the ultrasonic image forming unit 114 for forming the ultrasonic image on the basis of the ultrasonic waves received in the ultrasonic transmission/receipt unit 113, and the control unit 200 and the lid housing 120 including the display device (monitor 121) for displaying the ultrasonic image, and the lid housing 120 is attached to the main body housing 110, capable of being opened/closed, and the control unit 200 is provided with the automatic end portion 220 for ending the diagnostic ultrasound apparatus 100 (shutting off supply of power to the portable diagnostic ultrasound apparatus 100) when the lid housing 120 is closed and the safe end portion 224 for executing the safe end processing for safely ending the ongoing processing and processing scheduled to be executed in the portable diagnostic ultrasound apparatus 100.

The safe end portion 224 may execute the safe end processing after the end delay time which is the time determined in advance has elapsed since the lid housing 120 and the main body housing 110 are closed. Moreover, the safe end portion 224 may cancel the ongoing processing and the processing scheduled to be executed and hold the progress state at cancellation in association with the cancelled processing.

Moreover, if the previous end is the end by the automatic end portion, at startup of the portable diagnostic ultrasound apparatus, the control unit 200 may resume the processing held in association with the progress state from the state at cancellation.

Moreover, the automatic end portion 220 may store the examination information of the examination conducted in the portable diagnostic ultrasound apparatus immediately before the lid housing 120 is closed. The control unit 200 may restore the examination information stored by the automatic end portion 220 at the startup of the portable diagnostic ultrasound apparatus and conduct the examination in accordance with the restored examination information.

Moreover, the attitude detecting device 122 for detecting the attitude of the lid housing 120 after the lid housing 120 is closed may be further provided, and if it is detected that the attitude of the lid housing 120 is other than horizontal, the safe end portion 224 may execute the safe end processing immediately after the detection without waiting for elapse of the end delay time.

The automatic end portion 220 may be further provided with the automatic end notification portion 227 for notifying that the automatic end is to be executed at a predetermined time interval during the end delay time.

Moreover, the automatic end portion 220 may be so configured that, when the lid housing 120 is closed, supply of power to the ultrasonic transmission/receipt unit 113 and the display device (monitor 121) is shut off. Moreover, the automatic end portion 220 may be so configured that, when the lid housing 120 is opened during the end delay time, supply of power to the display device (monitor 121) is resumed.

Furthermore, the safe end portion 224 may be so configured that, for the ongoing processing, end of the processing is to be awaited in accordance with the user's setting.

Thus, according to this embodiment, deterioration of hardware caused by continuous electric conduction can be suppressed without increasing user workload as in the first embodiment or the second embodiment. Thus, safety of the apparatus is also improved.

Moreover, according to this embodiment, when the automatic end processing is to be executed, the user is notified of the fact. Therefore, the user can easily grasp that the automatic end is being executed.

### Fifth Embodiment

Subsequently, a fifth embodiment to which the present invention is applied will be explained. In this embodiment, if the portable diagnostic ultrasound apparatus 100 is driven by a battery, the end delay time T is adjusted by considering also a remaining battery capacity.

A configuration of the portable diagnostic ultrasound apparatus 100 of this embodiment is basically similar to that of the first embodiment. However, in this embodiment, as described above, the end delay time T is adjusted when the apparatus is driven by a battery, in accordance with the remaining battery capacity. Therefore, processing of the power supply control 225 and the automatic end portion 220 of this embodiment is different.

The power supply control portion 225 of this embodiment discriminates whether or not the power supply 112 is driven by a battery, and if power is supplied from a battery, when the user closes the lid housing 120, the remaining battery capacity (remaining battery capacity data) at that point of time is notified to the end condition setting portion 223. Whether or not the apparatus is driven by a battery is discriminated on the basis of whether or not an AC cable is connected, for example.

When the automatic end portion 220 receives the remaining battery capacity data from the power supply control portion 225 after receipt of the closed signal from the monitor opening/closing monitoring portion 221, the automatic end portion 220 calculates drivable time BT by the remaining battery capacity. The calculation of the drivable time BT is made by using loading. The loading is determined in advance and stored in the storage portion 230.

Then, the drivable time BT is compared with the set end delay time T, and if the drivable time BT is smaller (T>BT), the end delay time T is changed. The time to be changed is a value obtained by subtracting time required for safe end from the drivable time BT. The time required for the safe end is determined in advance and stored in the end condition storage portion 231. Then, after the end delay time T after the replacement has elapsed, the safe end is executed. If the drivable time BT is larger (T≦BT), the end delay time T is set as is.

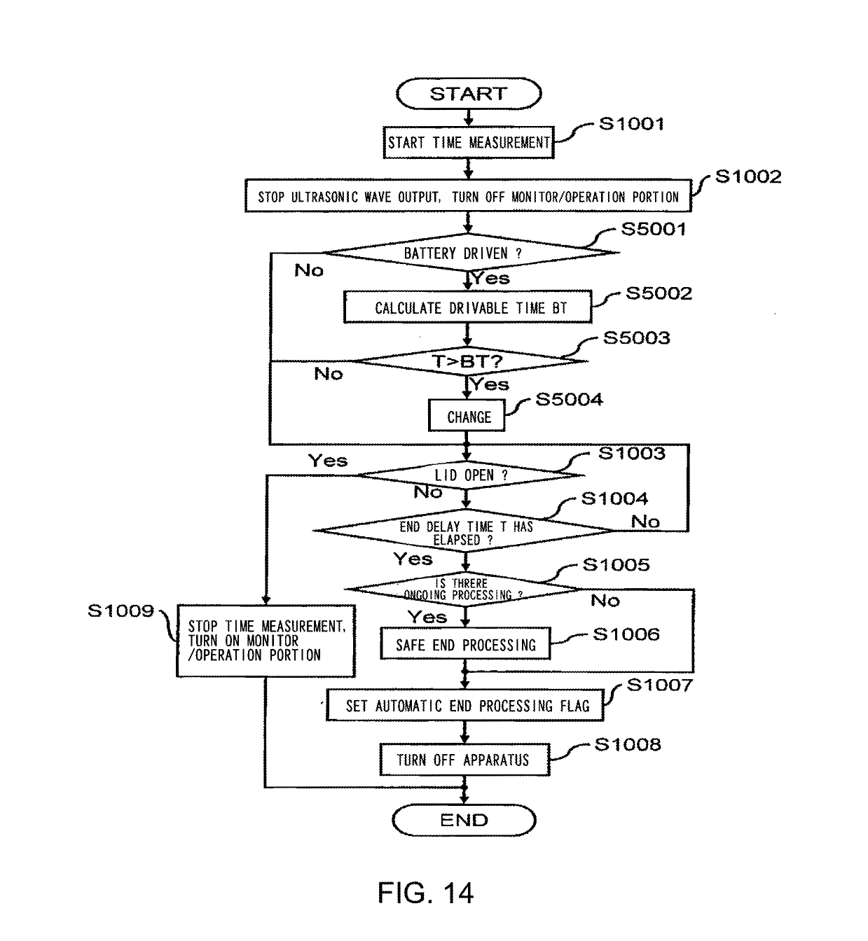

A flow of the automatic end processing by the automatic end portion 220 of this embodiment will be explained by reference to FIG. 14. The automatic end processing of this embodiment is basically similar to any one of the above described embodiments. Here, an instance basically similar to the first embodiment will be explained as an example.

Regarding the automatic end portion 220 of this embodiment, the time measuring portion 222 starts time measurement (Step S1001). Then, when the power supply control portion 225 stops output of ultrasonic waves and turns off the power supply of the monitor and the operation portion (Step S1002), it is discriminated whether or not the portable diagnostic ultrasound apparatus 100 is driven by a battery (Step S5001). The automatic end portion 220 makes this discrimination on whether or not the remaining battery capacity data is obtained from the power supply control portion 225. That is, if the remaining battery capacity data is received, it is discriminated that the apparatus is driven by a battery.

If the apparatus is driven by a battery, the automatic end portion 220 calculates the drivable time BT (Step S5002) and compares it with the end delay time T (Step S5003). If the drivable time BT is smaller (T>BT), the end delay time T is changed (Step S5004).

If the end delay time T is smaller in Step S5003, the processing in Step S5004 is not executed, and if the apparatus is not driven by a battery in Step S5001, processing in Steps S5002 to S5004 is not executed, but the routine proceeds to Step S1003.

The subsequent processing is similar to that of the first embodiment. The safe end processing and the start processing after the automatic end are also similar. Moreover, in this embodiment, the automatic end processing to serve as the basis may be the automatic end processing in the second embodiment, that in the third embodiment, or that in the fourth embodiment.

As described above, the portable diagnostic ultrasound apparatus 100 of this embodiment is the portable diagnostic ultrasound apparatus 100 provided with the main body housing 110 including the ultrasonic transmission/receipt unit 113 for generating and transmitting the ultrasonic waves to the ultrasonic probe and receiving the ultrasonic waves from the ultrasonic probe, the ultrasonic image forming unit 114 for forming the ultrasonic image on the basis of the ultrasonic waves received in the ultrasonic transmission/receipt unit 113, and the control unit 200 and the lid housing 120 including the display device (monitor 121) for displaying the ultrasonic image, and the lid housing 120 is attached to the main body housing 110, capable of being opened/closed, and the control unit 200 is provided with the automatic end portion 220 for ending the diagnostic ultrasound apparatus 100 (shutting off supply of power to the portable diagnostic ultrasound apparatus 100) when the lid housing 120 is closed and the safe end portion 224 for executing the safe end processing for safely ending the ongoing processing and the processing scheduled to be executed in the portable diagnostic ultrasound apparatus 100.

The safe end portion 224 may execute the safe end processing after the end delay time which is the time determined in advance has elapsed since the lid housing 120 and the main body housing 110 are closed. Moreover, the safe end portion 224 may cancel the ongoing processing and the processing scheduled to be executed and hold the progress state at cancellation in association with the cancelled processing.

Moreover, if the previous end is the end by the automatic end portion, at startup of the portable diagnostic ultrasound apparatus, the control unit 200 may resume the processing held in association with the progress state from the state at cancellation.

Moreover, the automatic end portion 220 may store the examination information of the examination conducted in the portable diagnostic ultrasound apparatus immediately before the lid housing 120 is closed. The control unit 200 may restore the examination information stored by the automatic end portion 220 at the startup of the portable diagnostic ultrasound apparatus and conduct the examination in accordance with the restored examination information.

Moreover, the attitude detecting device 122 for detecting the attitude of the lid housing 120 after the lid housing 120 is closed may be further provided, and if it is detected that the attitude of the lid housing 120 is other than horizontal, the safe end portion 224 may execute the safe end processing immediately after the detection without waiting for elapse of the end delay time.

The automatic end portion 220 may be further provided with the automatic end notification portion 227 for notifying that automatic end is to be executed at a predetermined time interval during the end delay time.

Moreover, the portable diagnostic ultrasound apparatus 100 is provided with a battery for supplying power to the portable diagnostic ultrasound apparatus, and the control unit 200 is further provided with a remaining capacity detecting portion (power supply control portion 225) for detecting a remaining battery capacity, and if the portable diagnostic ultrasound apparatus is driven by the battery, the automatic end portion 220 may compare the driving time by a remaining battery capacity with the end delay time and if the driving time is shorter, the end delay time may be changed in accordance with the driving time.

Moreover, the automatic end portion 220 may be so configured that, when the lid housing 120 is closed, supply of power to the ultrasonic transmission/receipt unit 113 and the display device (monitor 121) is shut off. Moreover, the automatic end portion 220 may be so configured that, when the lid housing 120 is opened during the end delay time, supply of power to the display device (monitor 121) is resumed.

Furthermore, the safe end portion 224 may be so configured that, for the ongoing processing, end of the processing is to be awaited in accordance with the user's setting.

Thus, according to this embodiment, deterioration of hardware caused by continuous electric conduction can be suppressed without increasing user workload, as in the first embodiment or the second embodiment. Thus, safety of the apparatus is also improved.

Moreover, according to this embodiment, in the case of battery-driven, the end delay time T is adjusted in accordance with the remaining battery capacity. Therefore, feasibility of the safe end processing in the automatic end processing of the portable diagnostic ultrasound apparatus 100 is improved, and safety is further improved.

### Sixth Embodiment

Subsequently, a sixth embodiment to which the present invention is applied will be explained. In this embodiment, by using the closing of the monitor 121 (lid housing 120) as a trigger, transfer of data (examination result) held in the storage portion 230 of the portable diagnostic ultrasound apparatus 100 to an external medium or to an external device is started in accordance with the user's setting. The data transfer is executed for a period until the end delay time T has elapsed.

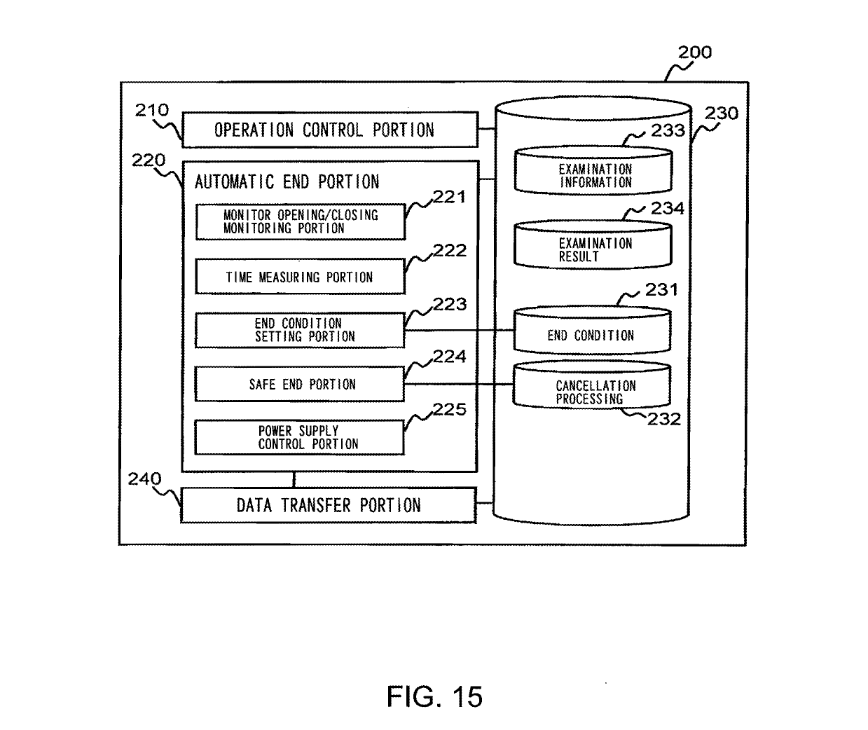

A configuration of the portable diagnostic ultrasound apparatus 100 of this embodiment is basically similar to that of the first embodiment. However, in this embodiment, as described above, data is transferred by using the closing of the monitor 121 as a trigger. Thus, the configuration of the control unit 200 of this embodiment is different. The functional block of the control unit 200 of this embodiment is illustrated in FIG. 15. As illustrated in this figure, the control unit 200 of this embodiment is provided with a data transfer portion 240 in addition to the configuration of the first embodiment illustrated in FIG. 2.

When the monitor 121 is closed, setting on whether or not to transfer data is set by the user through the operation portion 111. The end condition setting portion 223 stores the setting made through the operation portion 111 in the end condition storage portion 231. An initial value on presence of execution of data transfer may be set in advance in the end condition storage portion 231.

The data transfer portion 240 of this embodiment transfers the examination result stored in the examination result storage portion 234 to the outside by using the closing of the monitor 121 as a trigger. More specifically, when the monitor 121 is closed, it is discriminated whether or not the end condition has a setting to execute data transfer. If the setting is to execute data transfer, transfer preparation processing is executed. If it is discriminated that transfer is to be executed in the transfer preparation processing, data transfer is started.

The data transfer portion 240 of this embodiment uses information on whether or not the power supply 112 is driven by a battery in the transfer preparation processing. Therefore, in this embodiment, if the monitor 121 is closed, the power supply control portion 225 discriminates whether or not the power supply 112 is driven by a battery, similarly to the fifth embodiment, and transfers a discrimination result to the data transfer portion 140. If the discrimination result is battery-driven, the remaining battery capacity is also transferred to the data transfer portion 240.

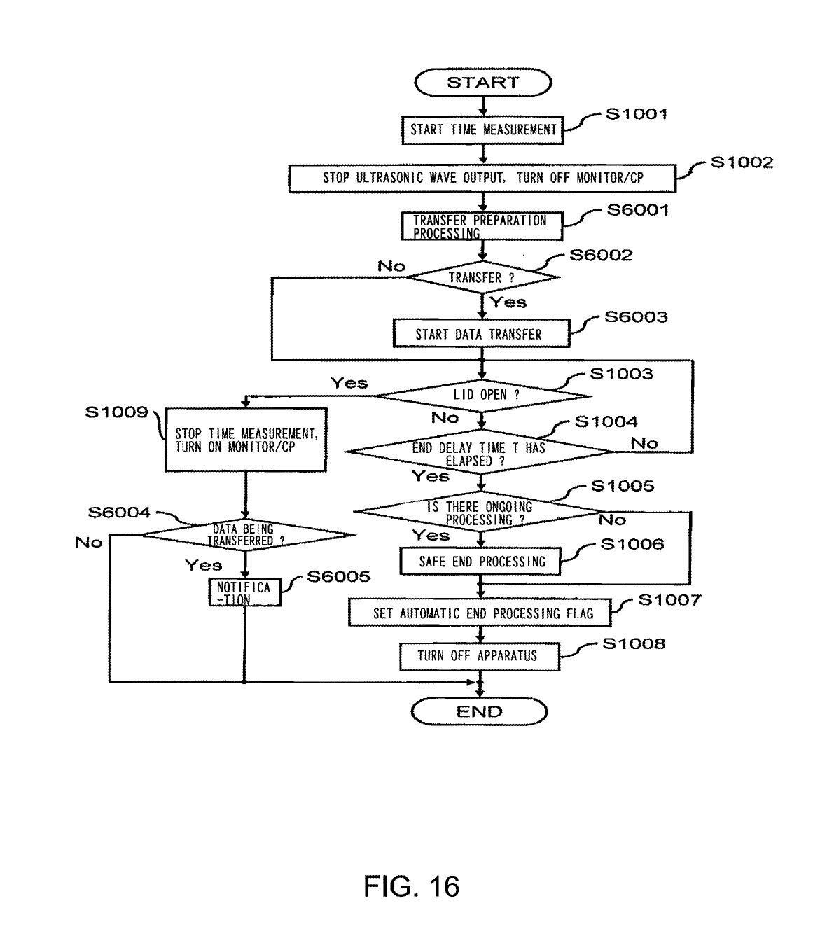

A flow of the automatic end processing by the automatic end portion 220 of this embodiment will be explained by reference to FIG. 16. The automatic end processing of this embodiment is basically similar to that of any one of the above-described embodiments. Here, the instance basically similar to that of the first embodiment will be explained as an example.

In this embodiment, when the time measuring portion 222 starts time measurement (Step S1001) and the power supply control portion 225 stops output of the ultrasonic waves and turns off the power supply of the monitor and the operation portion (Step S1002), the data transfer portion 240 executes the transfer preparation processing (Step S6001).

If it is determined that the data transfer is to be executed as the result of the transfer preparation processing (Step S6002), the data transfer portion 240 starts data transfer (Step S6003).

After that, in the automatic end portion 220, the processing of waiting for elapse of the end delay time T (Step S1004) and of executing the safe processing is similar to that of the first embodiment. If data transfer by the data transfer portion 240 is continuing at this point of time, it is determined to be ongoing processing in Step S1005. Moreover, while elapse of the end delay time T is awaited, if the monitor 121 is opened, if data is being transferred (Step S6004), the automatic end portion 220 notifies the user of the fact (Step S6005).

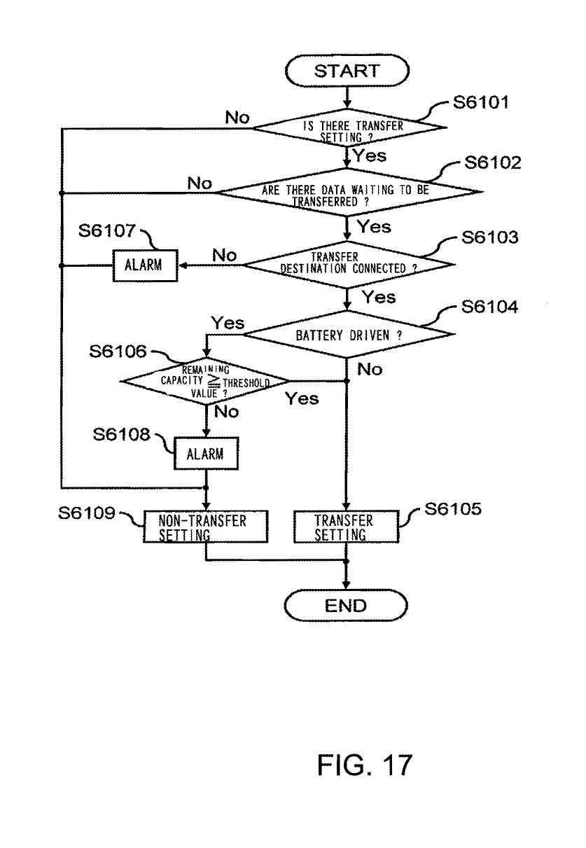

Subsequently, details of the transfer preparation processing by the data transfer portion 240 in Step S6001 of this embodiment will be explained. FIG. 17 shows a processing flow of the transfer preparation processing of this embodiment.

The data transfer portion 240 discriminates whether or not the setting is to transfer data when the monitor 121 is closed as the end condition (Step S6101). If the setting is to transfer data, it is discriminated whether or not data waiting to be transferred is held in the examination result storage portion 234 (Step S6102). If there is data waiting to be transferred, it is discriminated whether or not a transfer destination is connected (Step S6103).

Then, if the transfer destination is connected, the data transfer portion 240 discriminates whether or not the portable diagnostic ultrasound apparatus 100 is driven by a battery (Step S6104). Here, discrimination is made on the basis of information from the power supply control portion 225.

If the apparatus is not driven by a battery, the data transfer portion 240 effects setting that data transfer is to be executed (Step S6105). For example, a transfer flag is set.

On the other hand, in Step S6104, if it is discriminated that the apparatus is driven by a battery, the data transfer portion 240 discriminates whether or not the remaining battery capacity is at a threshold value determined in advance or more (Step S6106). The threshold value is determined in advance in accordance with data capacity that can be transferred in the end delay time T. It may be a fixed value or may be set for each of a plurality of end delay times T so that the value can be selected in accordance with the end delay time T. Moreover, it may be so configured that a plurality of values are set in advance in association with both a quantity of the data waiting to be transferred and the end delay time T, and a threshold value matching both conditions is selected.

If the remaining battery capacity is at the threshold value or more, the routine proceeds to Step S6105.

If transfer setting is not effected in Step S6101, if there is no data waiting to be transferred in Step S6102, if the transfer destination is not connected in Step S6103, and if the remaining capacity is smaller than the threshold value in Step S6106, the data transfer portion 240 sets such that the data transfer is not executed (Step S6109); for example, a non-transfer flag is set. At this time, if the transfer destination is not connected in Step S6103 or if the remaining capacity is smaller than the threshold value in Step S6106, the data transfer portion 240 givens an alarm on the sub monitor (Steps S6107, S6108). The alarm may be made in any method, such as display of a message on the sub monitor, flashing of an LED, output of a beep sound, and the like.

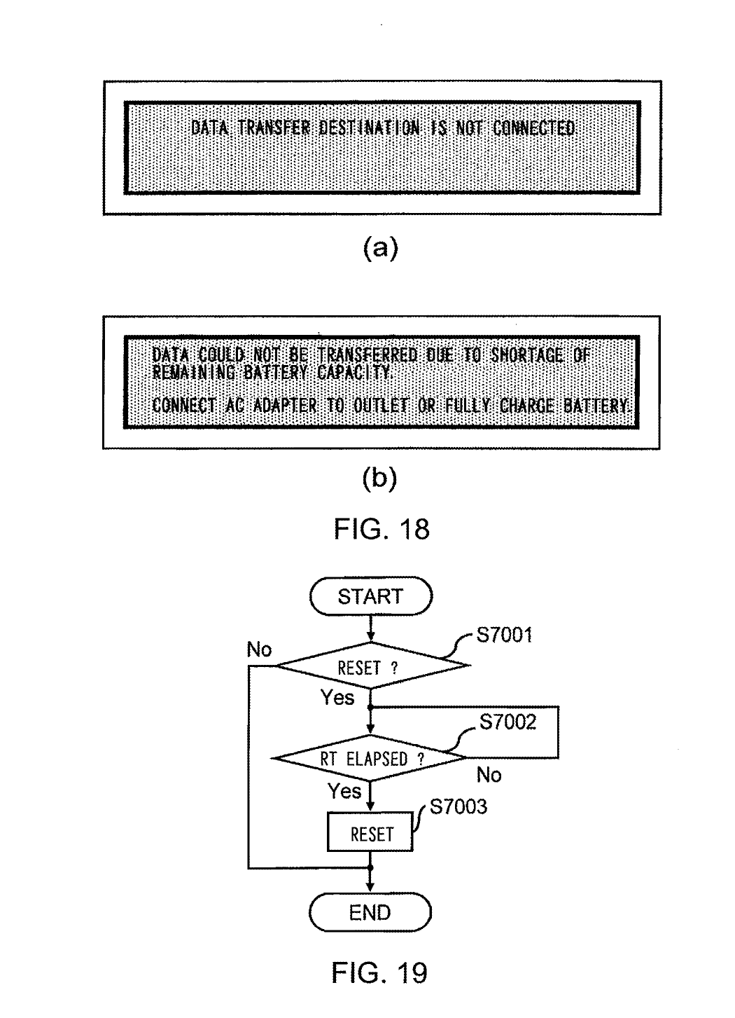

FIG. 18 illustrate examples of an alarm displayed on the sub monitor. FIG. 18(a) is an example message displayed in Step S6107, and FIG. 18(b) is an example message displayed is Step S6108. These messages may be configured to be displayed when the monitor 121 is opened immediately after the automatic end, for example.

In this embodiment, the instance in which, when the monitor 121 is closed, all the data stored in the storage portion 230 of the portable diagnostic ultrasound apparatus 100 is transferred is explained as an example, but this is not limiting. For example, a transfer capacity may be determined in advance. For example, the transfer capacity may be determined to be a capacity for one day, a capacity for one examination, or the like.

Moreover, it may be so configured that the transfer capacity is determined in accordance with the end delay time T. In this case, for example, in the transfer preparation processing, the data transfer portion 240 compares time (transfer time) TrT for transferring all the data to be transferred with the end delay time T. If the transfer time TrT is longer than the end delay time T (TrT>T), the data capacity that can be transferred within the end delay time T is calculated, and only the calculated data capacity is transferred.

In this embodiment, the safe end processing and the start processing after the automatic end are similar to those of the first embodiment. Moreover, in this embodiment, the automatic end processing to serve as the basis may be the automatic end processing of the second embodiment, that of the third embodiment, that of the fourth embodiment, or that of the fifth embodiment.

As described above, the portable diagnostic ultrasound apparatus 100 of this embodiment is the portable diagnostic ultrasound apparatus 100 provided with the main body housing 110 including the ultrasonic transmission/receipt unit 113 for generating and transmitting the ultrasonic waves to the ultrasonic probe and receiving the ultrasonic waves from the ultrasonic probe, the ultrasonic image forming unit 114 for forming the ultrasonic image on the basis of the ultrasonic waves received in the ultrasonic transmission/receipt unit 113, and the control unit 200 and the lid housing 120 including the display device (monitor 121) for displaying the ultrasonic image, and the lid housing 120 is attached to the main body housing 110, capable of being opened/closed, and the control unit 200 is provided with the automatic end portion 220 for ending the diagnostic ultrasound apparatus 100 (shutting off supply of power to the portable diagnostic ultrasound apparatus 100) when the lid housing 120 is closed and the safe end portion 224 for executing safe end processing for safely ending the ongoing processing and processing scheduled to be executed in the portable diagnostic ultrasound apparatus 100.

The safe end portion 224 may execute the safe end processing after the end delay time which is the time determined in advance has elapsed since the lid housing 120 and the main body housing 110 are closed. Moreover, the safe end portion 224 may cancel the ongoing processing and the processing scheduled to be executed and hold the progress state at cancellation in association with the cancelled processing.

Moreover, if the previous end is the end by the automatic end portion, at startup of the portable diagnostic ultrasound apparatus, the control unit 200 may resume the processing held in association with the progress state from the state at cancellation.

Moreover, the automatic end portion 220 may store the examination information of the examination conducted in the portable diagnostic ultrasound apparatus immediately before the lid housing 120 is closed. The control unit 200 may restore the examination information stored by the automatic end portion 220 at the startup of the portable diagnostic ultrasound apparatus and conduct the examination in accordance with the restored examination information.

Moreover, the attitude detecting device 122 for detecting the attitude of the lid housing 120 after the lid housing 120 is closed may be further provided, and if it is detected that the attitude of the lid housing 120 is other than horizontal, the safe end portion 224 may execute the safe end processing immediately after the detection without waiting for elapse of the end delay time.

The automatic end portion 220 may be further provided with the automatic end notification portion 227 for notifying that automatic end is to be executed at a predetermined time interval during the end delay time.

Moreover, if the portable diagnostic ultrasound apparatus 100 is provided with a battery for supplying power to the portable diagnostic ultrasound apparatus, the control unit 200 is further provided with a remaining capacity detecting portion (power supply control portion 225) for detecting a remaining battery capacity, and the portable diagnostic ultrasound apparatus is driven by the battery, the automatic end portion 220 may compare the driving time by a remaining battery capacity with the end delay time, and if the driving time is shorter, the end delay time may be changed in accordance with the driving time.

The examination result storage portion 234 for holding data obtained in the portable diagnostic ultrasound apparatus may be further provided, and the control unit 200 may be further provided with the data transfer portion 240 for transferring data held in the examination result storage portion 234 to the outside by using the closing of the lid housing 120 as a trigger.

Moreover, the automatic end portion 220 may be so configured that, when the lid housing 120 is closed, supply of power to the ultrasonic transmission/receipt unit 113 and the display device (monitor 121) is shut off. Moreover, the automatic end portion 220 may be so configured that, when the lid housing 120 is opened during the end delay time, supply of power to the display device (monitor 121) is resumed.

Furthermore, the safe end portion 224 may be so configured that, for the ongoing processing, end of the processing is to be awaited in accordance with the user's setting.

Thus, according to this embodiment, deterioration of hardware caused by continuous electric conduction can be suppressed without increasing user workload as in the first embodiment or the second embodiment. Thus, safety of the apparatus is improved.

Moreover, according to this embodiment, since it is so configured that data is transferred during the end delay time T by using the closing of the monitor 121 as a trigger, the images and measurement results obtained in the portable diagnostic ultrasound apparatus 100 can be reliably transferred to external media for storage without user workload. In the portable diagnostic ultrasound apparatus 100, since the external transfer of required data is executed within the end delay time T at the automatic end, the examination efficiency is also improved.

In each of the above described embodiments, the examination information stored in the examination information storage portion 233 may be reset after a predetermined time (reset time RT) has elapsed since the lid housing 120 is closed. In this case, setting of whether or not to reset the examination information of the examination after the reset time RT has elapsed since the lid housing 120 is closed and the reset time RT are set as end conditions.

The setting may be registered in advance in the end condition storage portion 231 or may be so configured that the operator inputs it through the operation portion 111, and the end condition setting portion 223 stores it in the end condition storage portion 231. An upper limit of the reset time RT is the end delay time T.

Regarding the automatic end processing in this case, only a portion relating to the reset processing is extracted and explained by reference to FIG. 19.

As the automatic end processing, in the automatic end portion 220, similarly to each of the above-described embodiments, when the monitor opening/closing monitoring portion 221 receives the closed signal, the time measuring portion 222 starts time measurement, and the power supply control portion 225 stops output of the ultrasonic waves and turns off the monitor 121 and the operation portion 111.

After that, the automatic end portion 220 discriminates whether or not the end condition is set such that the examination information is reset (Step S7001). If it is not set, the routine proceeds to Step S1003 as is.

On the other hand, if it is set, elapse of the reset time RT is awaited (Step S7002), the reset processing (Step 7003) is executed, and after the reset processing, the routine returns to Step S1003. If the monitor 121 (lid housing 120) is opened during this period, the routine proceeds to Step S1009 as usual.

As described above in each of the embodiments of the present invention, the automatic end portion 220 may be so configured that the examination information registered in the portable diagnostic ultrasound apparatus when the lid housing 120 is closed is reset after a predetermined time has elapsed since the lid is closed in accordance with the user's instruction.

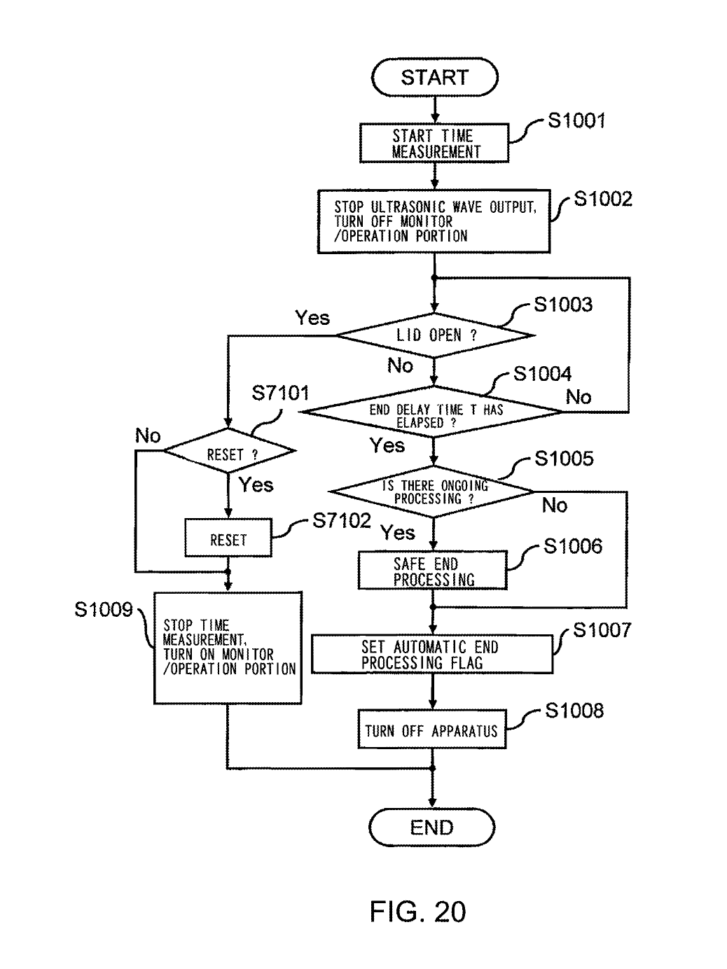

Moreover, on the contrary, it may be so configured that, if the monitor 121 (lid housing 120) is opened during the end delay time T, the examination information stored in the examination information storage portion 233 is reset. A flow of the automatic end processing in this case will be explained by reference to FIG. 20.

The flow of the automatic end processing in this case is basically similar to that of the first embodiment. However, if the monitor 121 (lid housing 120) is opened in Step S1003, the automatic end portion 220 discriminates whether or not the end condition is set such that the examination information is reset (Step S7101). If it is not set, the routine proceeds to Step S1009 as is.

On the other hand, if it is set, the reset processing (Step 7102) is executed, and after the reset processing, the routine proceeds to Step S1009.

Each of the above described-embodiments and variations can be applied not only to the portable diagnostic ultrasound apparatus but also to all diagnostic ultrasound apparatuses of the type in which the monitor is arranged in the lid housing and the monitor is accommodated in the main body housing when not in use.

### REFERENCE SIGNS LIST

100 portable diagnostic ultrasound apparatus
101 subject
110 main body housing
111 operation portion
112 power supply
113 ultrasonic transmission/receipt unit
114 ultrasonic image forming unit
120 lid housing
121 monitor
122 attitude detecting device
130 ultrasonic probe
131 cable
140 data transfer portion
200 control unit
210 operation control portion
220 automatic end portion
221 monitor opening/closing monitoring portion
222 time measuring portion
223 end condition setting portion
224 safe end portion
225 power supply control portion
227 time notification portion
230 storage portion
231 end condition storage portion
232 cancellation processing storage portion
233 examination information storage portion
234 examination result storage portion
240 data transfer portion

1. A portable diagnostic ultrasound apparatus comprising:
a main body housing including an ultrasonic transmission/receipt unit for generating and transmitting ultrasonic waves to an ultrasonic probe and for receiving the ultrasonic waves from the ultrasonic probe, an ultrasonic image forming unit for forming an ultrasonic image on the basis of the ultrasonic waves received in the ultrasonic transmission/receipt unit, and a control unit; and
a lid housing including a display device for displaying the ultrasonic image and attached to the main body housing capable of being opened/closed, wherein
the control unit is provided with an automatic end portion for ending the portable diagnostic ultrasound apparatus when the lid housing is closed; and
a safe end portion for executing safe end processing for safely ending ongoing processing and processing scheduled to be executed in the portable diagnostic ultrasound apparatus.

2. The portable diagnostic ultrasound apparatus according to claim 1, wherein
the safe end portion executes the safe end processing after an end delay time has elapsed, which is a time determined in advance since the lid housing is closed.

3. The portable diagnostic ultrasound apparatus according to claim 2, wherein
the safe end portion cancels the ongoing processing and the processing scheduled to be executed and holds a progress state at cancellation in association with the cancelled processing.

4. The portable diagnostic ultrasound apparatus according to claim 3, wherein
the control unit resumes the processing held in association with the progress state at startup of the portable diagnostic ultrasound apparatus from the state at cancellation.

5. The portable diagnostic ultrasound apparatus according to claim 3, wherein
the automatic end portion stores examination information of an examination conducted in the portable diagnostic ultrasound apparatus immediately before the lid housing is closed.

6. The portable diagnostic ultrasound apparatus according to claim 5, wherein
the control unit restores examination information stored by the automatic end portion at startup of the portable diagnostic ultrasound apparatus and conducts examination in accordance with the restored examination information.

7. The portable diagnostic ultrasound apparatus according to claim 2, further comprising:
an attitude detecting device for detecting an attitude of the lid housing after the lid housing is closed, wherein
if the attitude of the lid housing is detected to be other than horizontal, the safe end portion executes the safe end processing immediately after the detection without waiting for elapse of the end delay time.

8. The portable diagnostic ultrasound apparatus according to claim 2, wherein
the automatic end portion is further provided with an automatic end notification portion for notifying that automatic end is to be executed at a predetermined time interval during the end delay time.

9. The portable diagnostic ultrasound apparatus according to claim 1, further comprising:
a battery for supplying power to the portable diagnostic ultrasound apparatus, wherein
the control unit is further provided with a remaining capacity detecting portion for detecting a remaining capacity of the battery; and
the automatic end portion compares driving time by the remaining battery capacity with the end delay time if the portable diagnostic ultrasound apparatus is driven by the battery, and if the driving time is shorter, the end delay time is changed in accordance with the driving time.

10. The portable diagnostic ultrasound apparatus according to claim 2, further comprising:
an examination result storage portion for holding data obtained in the portable diagnostic ultrasound apparatus, wherein
the control unit is further provided with a data transfer portion for transferring the data held in the examination result storage portion to an outside medium or device by using the closing of the lid housing as a trigger.

11. The portable diagnostic ultrasound apparatus according to claim 1, wherein
the automatic end portion resets the examination information registered in the portable diagnostic ultrasound apparatus when the lid housing is closed in accordance with a user's instruction after a predetermined time has elapsed since the lid housing is closed.

12. The portable diagnostic ultrasound apparatus according to claim 1, wherein
the automatic end portion shuts down supply of power to the ultrasonic transmission/receipt unit and the display device when the lid housing is closed.

13. The portable diagnostic ultrasound apparatus according to claim 12, wherein
the automatic end portion resumes supply of power to the display device when the lid housing is opened during the end delay time.

14. The portable diagnostic ultrasound apparatus according to claim 3, wherein
the safe end portion waits for end of the processing for the ongoing processing in accordance with a user's setting.

15. A portable diagnostic ultrasound apparatus comprising:
a main body housing including an ultrasonic transmission/receipt unit for generating and transmitting ultrasonic waves to an ultrasonic probe and for receiving the ultrasonic waves from the ultrasonic probe, an ultrasonic image forming unit for forming an ultrasonic image on the basis of the ultrasonic waves received in the ultrasonic transmission/receipt unit, and a control unit; and
a lid housing including a display device for displaying the ultrasonic image and attached to the main body housing capable of being opened/closed, wherein
the control unit is provided with an automatic end portion for shutting off supply of power to the portable diagnostic ultrasound apparatus when the lid housing is closed; and
a safe end portion for executing safe end processing for safely ending ongoing processing and processing scheduled to be executed in the portable diagnostic ultrasound apparatus.
